(19) United States
(12) Patent Application Publication
(10) Pub. No.: US 2025/0049307 A1
(43) Pub. Date: Feb. 13, 2025

(54) STEERABLE INSTRUMENT FOR ENDOSCOPIC OR INVASIVE APPLICATIONS

(71) Applicant: FORTIMEDIX ASSETS II B.V., Geleen (NL)

(72) Inventor: Mattheus Hendrik Louis THISSEN, Swalmen (NL)

(73) Assignee: FORTIMEDIX ASSETS II B.V., Geleen (NL)

(21) Appl. No.: 18/718,821

(22) PCT Filed: Dec. 15, 2022
(86) PCT No.: PCT/NL2022/050721

§ 371 Date: Jun. 12, 2024

(30) Foreign Application Priority Data
Dec. 16, 2021 (NL) 2030159

(51) Int. Cl. A61B 1/005; A61B 1/00; A61M 25/00; A61M 25/01

(52) U.S. Cl. CPC A61B 1/0057; A61B 1/0011; A61M 25/0009; A61M 25/0147

(57) ABSTRACT

A steerable instrument has a tube (3) extending in a longitudinal direction. The steerable instrument has a proximal end and a deflectable distal end. The tube (3) has a steering wire (16(j); 120, 130) which is made from the tube (3), separated by a slotted structure from the remainder of the tube (3), attached to the deflectable distal end and is movable in a longitudinal direction of the tube (3) such as to deflect the deflectable distal end The steering wire (16(j); 120, 130) has flexible portion located in a flexible zone (13) of the steerable instrument which is implemented by a series of adjacent chain links (1301(k); 1401(k); 1501(k); 1601(k); 1701(k)).

### FIELD OF THE INVENTION

The present invention relates to a steerable instrument for endoscopic and/or invasive type of applications, such as in surgery. The steerable instrument according to the invention can be used in both medical and non-medical applications. Examples of the latter include inspection and/or repair of mechanical and/or electronic hardware at locations that are difficult to reach. Hence, terms used in the following description such as endoscopic application or invasive instrument, must be interpreted in a broad manner.

### BACKGROUND ART

Transformation of surgical interventions that require large incisions for exposing a target area into minimal invasive surgical interventions, i.e. requiring only natural orifices or small incisions for establishing access to the target area, is a well-known and ongoing process. In performing minimal invasive surgical interventions, an operator such as a physician, requires an access device that is arranged for introducing and guiding invasive instruments into the human or animal body via an access port of that body. In order to reduce scar tissue formation and pain to a human or animal patient, the access port is preferably provided by a single small incision in the skin and underlying tissue. In some applications, a natural orifice of the body can be used as an entrance. Furthermore, the access device preferably enables the operator to control one or more degrees of freedom that the invasive instruments offer. In this way, the operator can perform required actions at the target area in the human or animal body in an ergonomic and accurate manner with a reduced risk of clashing of the instruments used.

Surgical invasive instruments and endoscopes are well-known in the art. Both the invasive instruments and endoscopes can comprise a steerable tube that enhances its navigation and steering capabilities. Such a steerable tube may comprise a proximal end part including at least one flexible zone, a distal end part including at least one flexible zone, and an intermediate part, wherein the steerable tube further comprises a steering arrangement that is adapted for translating a deflection of at least a part of the proximal end part relative to the intermediate part into a related deflection of at least a part of the distal end part. Alternatively, the distal flexible zone may be steered by a robotic instrument arranged at the proximal end of the steerable instrument.

Steerable invasive instruments may comprise a handle that is arranged at the proximal end part of the steerable tube for steering the tube and/or for manipulating a tool that is arranged at the distal end part of the steerable tube. Such a tool can for example be a camera, a manual manipulator, e.g. a pair of scissors, forceps, or manipulators using an energy source, e.g. an electrical, ultrasonic or optical energy source.

Furthermore, such a steerable tube may comprise a number of co-axially arranged cylindrical elements including an outer cylindrical element, an inner cylindrical element and one or more intermediate cylindrical elements depending on the number of flexible zones in the proximal and distal end parts of the tube and the desired implementation of the steering members of the steering arrangement, i.e. all steering members can be arranged in a single intermediate cylindrical element or the steering members are divided in different sets and each set of steering members is arranged, at least in part, in a different or the same intermediate cylindrical element. In most prior art devices, the steering arrangement comprises conventional steering cables with, for instance, sub 1 mm diameters as steering members, wherein the steering cables are arranged between related flexible zones at the proximal and distal end parts of the tube. Other steering units at the proximal end, like ball shaped steering units or robot driven steering units, may be applied instead.

However, as steering cables have many well-known disadvantages, for some applications one may want to avoid them and to implement the steering members by one or more sets of longitudinal steering elements that form integral parts of the one or more intermediate cylindrical elements. Each of the intermediate cylindrical elements including the longitudinal steering elements, i.e., the steering wires, can be fabricated either by using a suitable material addition technique, such as injection molding or plating, or by starting from a tube and then using a suitable material removal technique, such as laser cutting, photochemical etching, deep pressing, conventional chipping techniques such as drilling or milling or high-pressure water jet cutting systems. Longitudinal steering elements manufactured in that way are, then, implemented as longitudinal strips resulting from the tube material, and can be used as pulling/pushing wires. Of the aforementioned material removal techniques, laser cutting is very advantageous as it allows a very accurate and clean removal of material under reasonable economic conditions.

The inner and outer cylindrical elements may be manufactured from tubes too. These tubes should be flexible at locations where the distal end, and possibly the proximal end too, of the instrument is bendable. Also at other locations where the instrument should be flexible, the inner and outer cylindrical elements should be flexible. This can be implemented by providing the inner and outer cylindrical elements with hinges at these flexible locations. Such hinges may result from (laser) cutting predetermined patterns in the tube. Many different patterns are known from the prior art. Which pattern to use depends on design requirements at the location concerned including but not limited to the required bending angle, bending flexibility, longitudinal stiffness, and radial stiffness.

Further details regarding the design and fabrication of the abovementioned steerable tube and the steering arrangement thereof have been described for example in WO 2009/112060 A1, WO 2009/127236 A1, U.S. Ser. No. 13/160,949, and U.S. Ser. No. 13/548,935 of the applicant, all of which are hereby incorporated by reference in their entirety.

Many steerable instruments have in common that manipulation of the user interface, for example a handle or a joystick, is transformed into longitudinal displacement of longitudinal steering elements, that run from the user interface end of the instrument to the tip of the instrument. At the tip, this displacement is transformed into steering of that tip. In most cases, steering of the tip is accomplished by bending of the tip. A common problem is that the tip of the instrument has a certain resistance against that bending. This bending stiffness is caused by for example the bending stiffness of the used construction of the flexible zone in the tip, the actuation cable of the tool attached to the tip, the guiding tube of this cable, the electrical isolation around the flexible part of the tip, etc., but also by the bending stiffness of the steering elements itself. This is cause of many disadvantages.

When the tip has almost no bending stiffness, only small forces in the longitudinal steering elements are required to bend the tip and the biggest part of the applied forces to the steering elements by the user, can be used for tissue manipulation forces. When the tip has an increased bending stiffness, bigger forces are needed to bend the tip and less of the forces can be used for tissue manipulation at a certain mechanical strength of the tip and steering elements construction. When this residual tissue manipulation force is not sufficient for adequate use of the instrument, one can only increase this force by using a stronger construction with stronger steering elements. This on itself usually also increases the stiffness of the construction and one ends up with the same problem. The right solution for this problem is often a trade-off between many performance aspects like the achievable bending angle, tissue manipulation forces (often referred to as ‘payload’ of the instrument), haptic feedback (the bending force can be that high that the user mainly ‘feels’ bending force and the tissue manipulation force is fully camouflaged by the required steering force), the achievable fatigue life (how many times can the tip bend before failure of the construction or the steering elements), etc.

It is clear that optimizing an instrument's performance often is achieved by minimizing the bending stiffness of the flexible part of the tip. Many solutions have been found. One can use for example a less stiff construction of coils or hinges instead of a flexible tube for the body of the flexible section or for the guiding tube of the tip tool actuation cable. One can also use an actuation cable for the tip tool that is as flexible as possible, for example, a cable of stranded metal wires is more flexible than a solid metal wire. Also for the steering elements one can use these stranded cables or for example Dyneema, Aramide or other high strength fibers.

Also in steerable instruments that are made from solid material tubes, like as in WO 2009/112060 A1, WO 2009/127236 A1 stiffness of the steering elements may be improved. Because the steering elements are of a solid metal they are already more stiff than stranded cables.

EP2259710A discloses steerable instruments in which steering wires are made by cutting strips from a tube. In an embodiment, portions of the steering wire at the proximal end and at the distal end are made from another material than the rest of the steering wire. The rest of the steering wire is connected to the proximal and distal portions by interconnecting joints. Such interconnection joints are only applied in portions of the steering wire which do not coincide with longitudinal locations of the instruments that have to bend or deflect. They are not designed to be flexible themselves.

WO 2010/151698 A2 describes a steerable medical delivery device, including a steerable portion comprising a first tubular member and a second tubular member, wherein one of the first and second tubular members is disposed within the other, wherein the first and second tubular elements are axially fixed relative to one another at a fixation location distal to the steerable portion, and wherein the first and second tubular members are axially movable relative to one another along the steerable portion to steer the steerable portion in a first direction, and wherein the first tubular member is adapted to preferentially bend in a first direction. In this known device each tube has a spine in a bendable region of the device. In some embodiments the spine is implemented by means of adjacent portions of the tube wherein the portions are provided with extensions located inside a hole of an adjacent portion such that adjacent portions cannot move relative to one another in the axial direction of the device. However, these adjacent portions are attached to one another by a spiral strip, and adjacent portions are shaped such that they cannot rotate relative to one another in the tangential direction of the tube.

### SUMMARY OF THE INVENTION

It is an object of the invention to provide a steerable instrument for endoscopic and/or invasive type of applications having steering wires with an improved balance between strength and bending stiffness.

In particular, it is an object of the invention to provide a steerable instrument having steering wires in the form of strips resulting from cutting predetermined patterns in a tube which strips having an optimized strength and a bending stiffness along their length.

More specifically, by implementing at least those parts of steering wires which need to be flexible in the form of shackled chain links which are resulting from cutting patterns in such tubes too, these flexible steering wire parts can have a minimum bending stiffness. This is especially advantageous in those parts of the instrument that need to be deflected in use as controlled from the proximal end.

To that end, independent aspects of the invention are defined in independent claims whereas dependent claims relate to advantageous embodiments.

According to a first aspect, defined in independent claim 1, a steerable instrument is provided comprising at least one tube extending in a longitudinal direction, the steerable instrument having a proximal end and a deflectable distal end, the at least one tube comprising at least one steering wire which is made from the at least one tube, separated by a slotted structure from the remainder of the at least one tube, attached to the deflectable distal end and configured to be movable in a longitudinal direction of the at least one tube such as to deflect the deflectable distal end, the at least one steering wire having at least one flexible portion located in a flexible zone of the steerable instrument and implemented by a series of adjacent chain links.

The at least one steering wire can be formed by cutting predetermined patterns in the tube, e.g. through a materials removing technique, thereby forming the slotted structure. Each steering wire of the at least one steering wire may be implemented as a chain link in the flexible portion, extending in the longitudinal direction and separated laterally from other steering wires and/or from the remainder of the tube. The at least one steering wire is separated, in a circumferential direction of the tube, from adjacent steering wires and from eventual remaining portions of the tube not forming part of a steering wire. Each of the at least one steering wire may hence be seen as forming a free-standing structure, attached to the deflectable distal end and extending in the longitudinal direction of the tube.

The slotted structure may comprise slots extending parallel to and along lateral sides of the at least one steering wire.

Hence, by the slotted structure separating the steering wire from the remainder of the tube, the steering wire is not connected nor attached laterally to other parts or portions of the tube. In particular, in the flexible zone, where the steering wire is implemented as a series of adjacent chain links, these chain links are linked, connected or attached, to adjacent chain links to form a chain forming at least a portion of the steering wire. Due to the slotted structure, the chain links are not connected nor attached to other elements or portions of the tube, in particular not to other tube portions in a direction different from the longitudinal direction, thus making the bendable region more flexible than in e.g. the prior art device of WO 2010/151698.

The expression of the steering wire and/or chain link as “extending in the longitudinal direction” encompasses both an extension parallel to an axial direction of the tube, as well as an extension in a helical manner along the tube, wherein the center of the helix substantially coincides with the axis of the tube.

In this application, the terms “proximal” and “distal” are defined with respect to an operator, e.g. a robot or physician that operates the instrument or endoscope. For example, a proximal end part is to be construed as a part that is located near the robot or physician and a distal end part as a part located at a distance from the robot or physician, i.e., in the area of operation.

### BRIEF DESCRIPTION OF THE DRAWINGS

Further features and advantages of the invention will become apparent from the description of the invention by way of non-limiting and non-exclusive embodiments. These embodiments are not to be construed as limiting the scope of protection. The person skilled in the art will realize that other alternatives and equivalent embodiments of the invention can be conceived and reduced to practice without departing from the scope of the present invention. Embodiments of the invention will be described with reference to the figures of the accompanying drawings, in which like or same reference symbols denote like, same or corresponding parts, and in which:

FIG. 1 shows a schematic cross sectional view of an invasive instrument assembly according to the prior art having one bendable distal end portion and one proximal end portion which controls the bending of the bendable distal end portion by means of strips cut out in a cylindrical element.

FIG. 2 shows a schematic overview of three cylindrical elements from which the instrument of FIG. 1 may be manufactured.

FIG. 3a shows a portion of an intermediate cylindrical element of the instrument of FIGS. 1 and 2.

FIG. 3b shows an alternative example of an intermediate cylindrical element of such an instrument.

FIG. 4 shows an example intermediate cylindrical element and an inner cylindrical element inserted in the intermediate cylindrical element.

FIG. 5 shows an outside view of a steerable invasive instrument assemble according to the prior art having two steerable bendable distal end portions and two proximal flexible control portions.

FIG. 6 shows an enlarged view of the distal tip of the instrument shown in FIG. 5.

FIG. 7 shows a cross section view through the invasive instrument shown in FIG. 5.

FIGS. 8 and 9 show examples of how the invasive instrument of FIGS. 5 and 7 can bend.

FIG. 10 shows a prior art example of a steerable invasive instrument assembly having two steerable bendable distal end portions and two proximal flexible control portions, as well as a flexible zone in between.

FIGS. 11A and 11B show prior art examples of a proximal end portion of a steerable invasive instrument with steering wires in the form of strips which can be coupled to a robotic controller configured to control movement of the steering wires.

FIGS. 12A, 12B and 12C show some prior art examples of series of chain links.

FIGS. 13A and 13B show an example of a series of chain links cut from a tube.

FIGS. 14A, 14B show examples of principles of chain links.

FIGS. 15A, 15B, 16A, 16B, 17A, 17B, 19A, 19B, 20A, 20B, 21A, 21B, 22, 23A, 23B show further examples of a series of chain links cut from a tube.

FIG. 18 shows a cross section of an instrument according to an embodiment.

FIG. 24 shows how fracture elements can be used in the manufacturing process of embodiments of the invention.

FIGS. 25 and 26 show further examples of a series of chain links cut from a tube in which adjacent chain links are attached to one another by flexible bridges.

FIGS. 27A, 27B, 27C show an example of an instrument in which a series of chain links is applied.

FIGS. 28, 29A, 29B, 30 show examples of an embodiment in which freely rotatable force equalizing structures are used.

### DESCRIPTION OF EMBODIMENTS

For the purpose of the present document, the terms cylindrical element and tube may be used interchangeably, i.e., like the term tube a cylindrical element also refers to a physical entity. The invention will be explained with reference to longitudinal steering elements which are cut from such cylindrical elements and are operative as push and/or pull wires to transfer movement of the steering elements at the proximal end of the instrument to the distal end to thereby control bending of one or more flexible distal end portions.

Instruments in which the Invention can be Applied

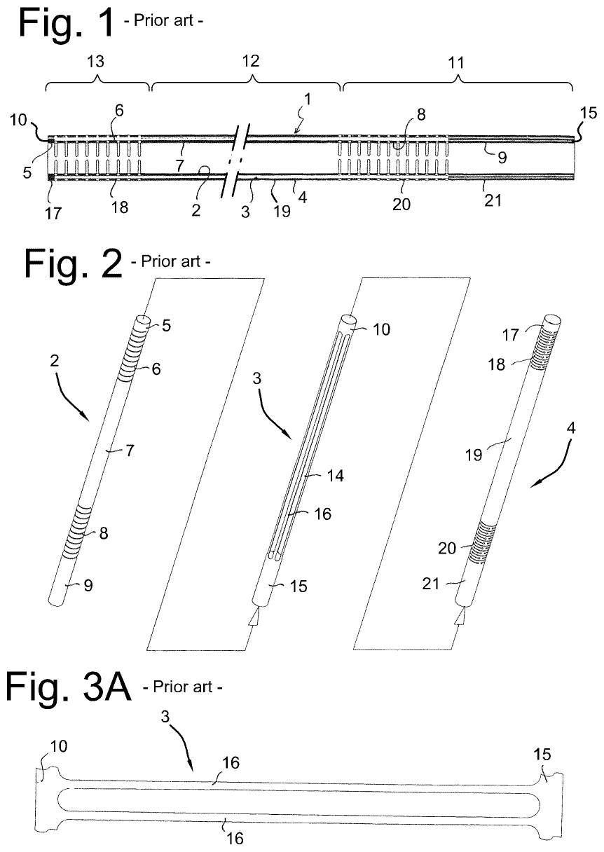

FIGS. 1, 2, 3a, and 3b are known from WO2009/112060. They are explained in detail because the present invention can be applied in this type of instruments.

FIG. 1 shows a longitudinal cross-section of a prior art steerable instrument comprising three co-axially arranged cylindrical elements, i.e. inner cylindrical element 2, intermediate cylindrical element 3 and outer cylindrical element 4. Suitable materials to be used for making the cylindrical elements 2, 3, and 4 include stainless steel, cobalt-chromium, shape memory alloy such as Nitinol®, plastic, polymer, composites or other materials that can be shaped by material removal processes like laser cutting or EDM. Alternatively, the cylindrical elements can be made by a 3D printing process or other known material deposition processes.

The inner cylindrical element 2 comprises a first rigid end part 5, which is located at a distal end part 13 of the instrument, a first flexible part 6, an intermediate rigid part 7 located at an intermediate part 12 of the instrument, a second flexible part 8 and a second rigid end part 9, which is located at a proximal end part 11 of the instrument. Distal end part 13 is a distal deflectable zone 13. Proximal end part 11 is a proximal bendable zone 11.

The outer cylindrical element 4 also comprises a first rigid end part 17, a first flexible part 18, an intermediate rigid part 19, a second flexible part 20 and a second rigid end part 21. The lengths of the parts 5, 6, 7, 8, and 9, respectively, of the cylindrical element 2 and the parts 17, 18, 19, 20, and 21, respectively, of the cylindrical element 4 are, preferably, substantially the same so that when the inner cylindrical element 2 is inserted into the outer cylindrical element 4, these different respective parts are longitudinally aligned with each other.

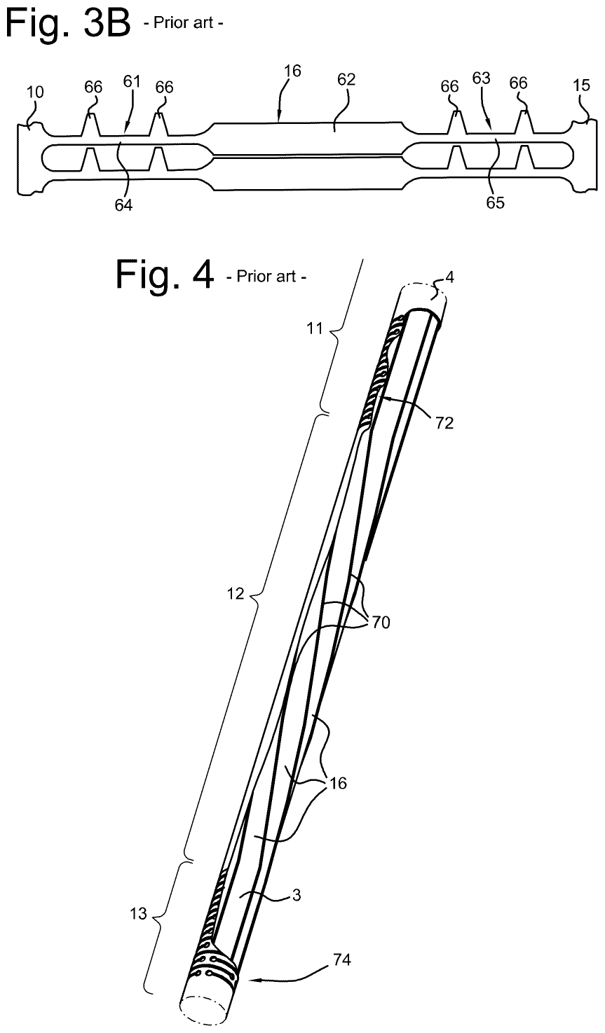

The intermediate cylindrical element 3 also has a first rigid end part 10 and a second rigid end part 15 which in the assembled condition are located between the corresponding rigid parts 5, 17 and 9, 21 respectively of the two other cylindrical elements 2, 4. The intermediate part 14 of the intermediate cylindrical element 3 comprises one or more separate longitudinal steering wires 16 which can have different forms and shapes as will be explained below. In FIG. 3a, two such longitudinal steering wires 16 are shown. After assembly of the three cylindrical elements 2, 3 and 4 whereby the element 2 is inserted in the element 3 and the two combined elements 2, 3 are inserted into the element 4, at least the first rigid end part 5 of the inner cylindrical element 2, the first rigid end part 10 of the intermediate cylindrical element 3 and the first rigid end part 17 of the outer cylindrical element 4 at the distal end of the instrument are attached to each other, e.g., by means of glue or one or more laser welding spots. In the embodiment shown in FIGS. 1 and 2, also the second rigid end part 9 of the inner cylindrical element 2, the second rigid end part 15 of the intermediate cylindrical element 3 and the second rigid end part 21 of the outer cylindrical element 4 at the proximal end of the instrument are attached to each other, e.g. by means of glue or one or more laser welding spots, such that the three cylindrical elements 2, 3, 4 form one integral unit.

In the embodiment shown in FIG. 2 the intermediate part 14 of intermediate cylindrical element 3 comprises a number of longitudinal steering wires 16 with a uniform cross-section so that the intermediate part 14 has the general shape and form as shown in the unrolled condition of the intermediate cylindrical element 3 in FIG. 3a. From FIG. 3a it also becomes clear that the intermediate part 14 is formed by a number of over the circumference of the intermediate cylindrical part 3, possibly equally, spaced parallel longitudinal steering wires 16. Advantageously, the number of longitudinal steering wires 16 is at least three, so that the instrument becomes fully controllable in any direction, but any higher number is possible as well. The number of longitudinal steering wires 16 may, e.g., be six or eight.

It is observed that the longitudinal steering wires 16 need not have a uniform cross section across their entire length. They may have a varying width along their length, possibly such that at one or more locations adjacent longitudinal steering wires 16 are only separated by a small slot resulting from the laser cutting in the cylindrical element 3. These wider portions of the longitudinal steering wires, then, operate as spacers to prevent adjacent longitudinal steering wires 16 from buckling in a tangential direction in a pushed state. Spacers may, alternatively, be implemented in other ways.

An embodiment with spacers is shown in FIG. 3b which shows two adjacent longitudinal steering wires 16 in an unrolled condition. In the embodiment shown in FIG. 3b each longitudinal steering wire 16 is composed of three portions 61, 62 and 63, co-existing with the first flexible part 6, 18 the intermediate rigid part 7, 19 and the second flexible part 8, 20 respectively. In the portion 62 coinciding with the intermediate rigid portion, each pair of adjacent longitudinal steering wires 16 is almost touching each other in the tangential direction so that in fact only a narrow slot is present there between just sufficient to allow independent movement of each longitudinal steering wire. The slot results from the manufacturing process and its width is, e.g., caused by the diameter of a laser beam cutting the slot.

In the other two portions 61 and 63 each longitudinal steering wire consists of a relatively small and flexible part 64, 65 as seen in circumferential direction, so that there is a substantial gap between each pair of adjacent flexible parts, and each flexible part 64, 65 is provided with a number of spacers 66, extending in the tangential direction and almost bridging completely the gap to the adjacent flexible part 64, 65. Because of these spacers 66 the tendency of the longitudinal steering wires 16 in the flexible portions of the instrument to shift in tangential direction is suppressed and tangential direction control is improved. The exact shape of these spacers 66 is not very critical, provided they do not compromise flexibility of flexible parts 64 and 65. The spacers 66 may or may not form an integral part with the flexible parts 64, 65 and may result from a suitable laser cutting process too.

In the embodiment shown in FIG. 3b the spacers 66 are extending towards one tangential direction as seen from the flexible part 64, 65 to which they are attached. It is however also possible to have these spacers 66 extending to both circumferential directions starting from one flexible part 64, 65. By using this it is either possible to have alternating types of flexible parts 64, 65 as seen along the tangential direction, wherein a first type is provided at both sides with spacers 66 extending until the next flexible part, and a second intermediate set of flexible parts 64, 65 without spacers 66. Otherwise it is possible to have flexible parts with cams at both sides, where as seen along the longitudinal direction of the instrument the cams originating from one flexible part are alternating with spacers originating from the adjacent flexible parts. It is obvious that numerous alternatives are available.

In the embodiment of FIGS. 1-3b, the steering wires 16 are attached to both the distal end and the proximal end of the instrument. Once an operator (or robotic device) bends proximal bendable zone 11 the steering wires 16 will move in the longitudinal direction of the instrument. The direction of longitudinal movement depends on the proximal bending direction of bendable zone 11. Some of the steering wires 16 may move in the proximal direction whereas tangentially opposite steering wires will move in the distal direction. This will cause the distal bendable zone 13 to deflect in the same clock-wise or anti clock-wise direction as proximal bendable zone 11.

The production of such an intermediate part is most conveniently done by injection moulding or plating techniques or starting from a cylindrical tube with the desired inner and outer diameters and removing parts of the wall of the cylindrical tube required e.g. by laser or water cutting to end up with the desired shape of the intermediate cylindrical element 3. However, alternatively, any 3D printing method can be used.

The removal of material can be done by means of different techniques such as laser cutting, photochemical etching, deep pressing, conventional chipping techniques such as drilling or milling, high pressure water jet cutting systems or any suitable material removing process available. Preferably, laser cutting is used as this allows for a very accurate and clean removal of material under reasonable economic conditions. The above mentioned processes are convenient ways as the cylindrical element 3 can be made so to say in one process, without requiring additional steps for connecting the different parts of the intermediate cylindrical element as required in the conventional instruments, where conventional steering cables must be connected in some way to the end parts. The same type of technology can be used for producing the inner and outer cylindrical elements 2 and 4 with their respective flexible parts 6, 8, 18 and 20. These flexible parts 6, 8, 18 and 20 can be manufactured as hinges resulting from cutting out any desired pattern from the cylindrical elements, e.g., by using any of the methods described in European patent application 08 004 373.0 filed on 10 Mar. 2008, page 5, lines 15-26, but any other suitable process can be used to make flexible portions.

It is observed that the instruments shown in FIGS. 4-10 are known from prior art WO2020/214027. Also in these instruments the present invention can be applied.

FIG. 4 shows an exemplary embodiment of steering wires 16 that have been obtained after providing longitudinal slots 70 to the wall of the intermediate cylindrical element 3 that interconnects proximal flexible zone 11 and distal flexible zone 13 as described above. Here, longitudinal steering wires 16 are, at least in part, spiraling about a longitudinal axis of the instrument such that an end portion of a respective steering wire 16 at the proximal portion of the instrument is arranged at another angular orientation about the longitudinal axis than an end portion of the same longitudinal steering wire 16 at the distal portion of the instrument. Were the longitudinal steering wires 16 arranged in a linear orientation, than a bending of the instrument at the proximal portion in a certain plane would result in a bending of the instrument at the distal portion in the same plane but in a 180 degrees opposite direction. This spiral construction of the longitudinal steering wires 16 allows for the effect that bending of the instrument at the proximal portion in a certain plane may result in a bending of the instrument at the distal portion in another plane, or in the same plane in the same direction. A preferred spiral construction may be such that the end portion of a respective steering wire 16 at the proximal portion of the instrument is arranged at an angularly shifted orientation of 180 degrees about the longitudinal axis relative to the end portion of the same longitudinal steering wire 16 at the distal portion of the instrument. However, e.g. any other angularly shifted orientation, e.g. 90 degrees, is within the scope of this document. The slots 70 are dimensioned such that movement of a longitudinal steering wire is guided by adjacent longitudinal steering wires when provided in place in a steerable instrument. However, especially at the flexible zones 11, 13 of the instrument, the width of longitudinal steering wires 16 may be less to provide the instrument with the required flexibility/bendability at those locations.

Whereas our cylindrical element 4 and inner cylindrical element 2, respectively, as shown in FIGS. 1 and 2, have rigid parts 19 and 7, respectively, these rigid parts 19, 7 may be provided with one or more suitable flexible parts in zone 12. This may be implemented by providing rigid parts 19, 7 with one or more slotted structures to provide intermediate cylindrical element with a desired flexibility. The longitudinal length of the slotted structure in flexible zone 77 depends on the desired application. Applications that may require one or more such extra flexible zones (of which the bending is not controlled from the proximal end) are endoluminal applications or surgery in the stomach, heart, lung, etc.

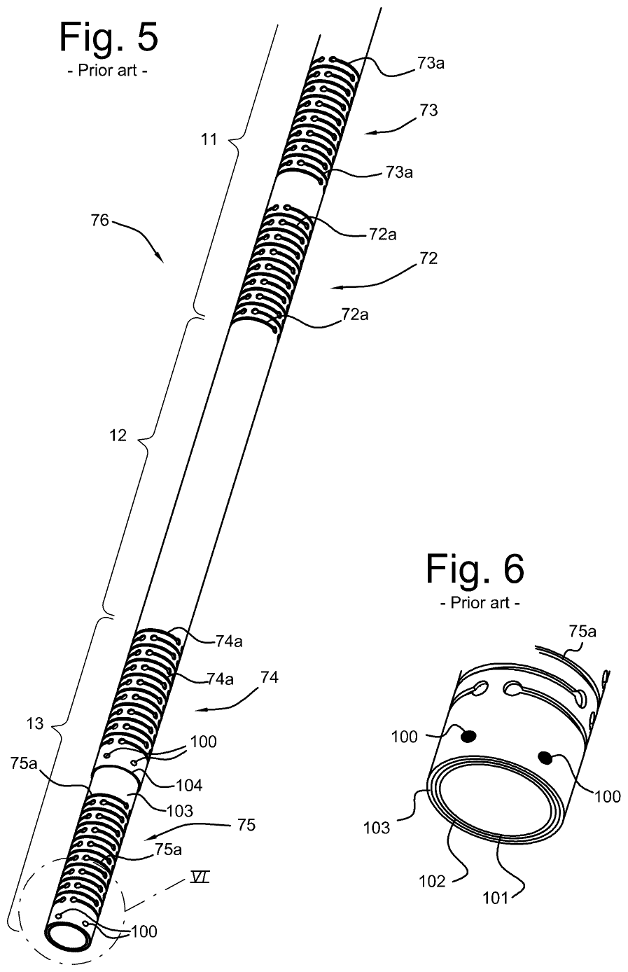

FIG. 5 provides a detailed perspective view of the distal portion of a prior art embodiment of an elongated tubular body 76 of a steerable instrument which has two steerable distal bendable zones 74, 75 which are operated by two bendable proximal zones 72, 73, respectively. FIG. 5 shows that the elongated tubular body 76 comprises a number of co-axially arranged layers or cylindrical elements including an outer cylindrical element 104 that ends after a first distal flexible zone 74 at the distal end portion 13. The distal end portion 13 of the outer cylindrical element 104 is fixedly attached to a cylindrical element 103 located inside of and adjacent to the outer cylindrical element 104, e.g. by means of (laser) spot welding at welding spots 100. However, any other suitable attachment method can be used, including any mechanical snap fit connection or gluing by a suitable glue.

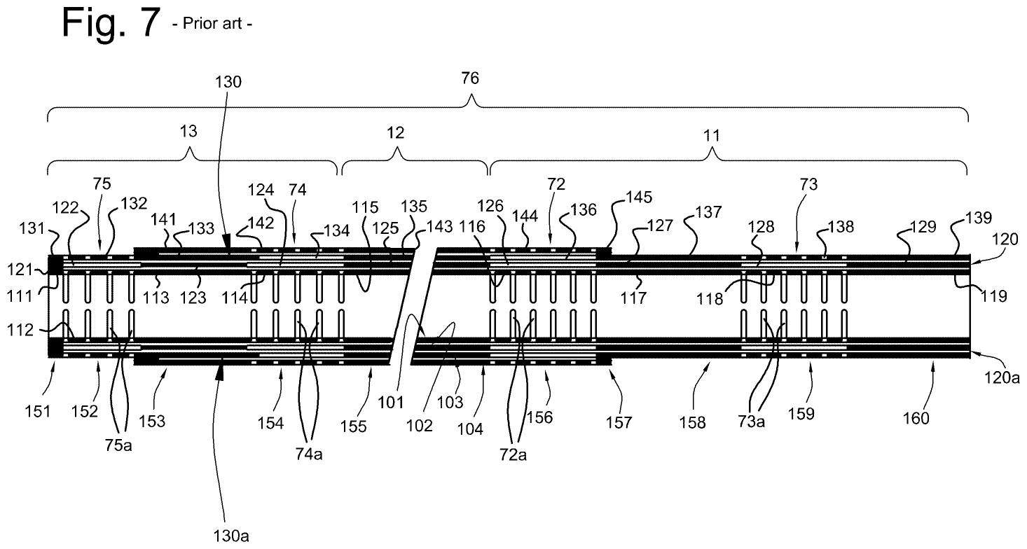

FIG. 6 provides a more detailed view of the distal end part 13 and shows that, in this embodiment, it includes three co-axially arranged layers or cylindrical elements, i.e., an inner cylindrical element 101, a first intermediate cylindrical element 102 and a second intermediate cylindrical element 103. The distal ends of inner cylindrical element 101, first intermediate cylindrical element 102 and second intermediate cylindrical element 103 are all three fixedly attached to one another. This may be done by means of (laser) spot welding at welding spots 100. However, any other suitable attachment method can be used, including any mechanical snap fit connection or gluing by a suitable glue. The points of attachment may be at the end edges of inner cylindrical element 101, first intermediate cylindrical element 102 and second intermediate cylindrical element 103, as shown in the figures. However, these points of attachment may also be located some distance away from these edges, be it, preferably, between the end edges and the locations of the flexible zone 75.

It will be clear to the skilled person that the elongated tubular body 76 as shown in FIG. 5 comprises four cylindrical elements in total. The elongated tubular body 76 according to the embodiment shown in FIG. 5 comprises two intermediate cylindrical elements 102 and 103 in which the steering members of the steering arrangement are arranged. However, extra or less cylindrical elements may be provided if desired. Steering wires may e.g., be arranged in and made from a single tube.

The steering arrangement in the exemplary embodiment of the elongated tubular body 76 as shown in FIG. 5 comprises the two flexible zones 72, 73 at the proximal end part 11 of the elongated tubular body 76, the two flexible zones 74, 75 at the distal end part 13 of the elongated tubular body 76 and the steering members that are arranged between related flexible zones at the proximal 11 and distal 13 end parts. An exemplary actual arrangement of the steering members is shown in FIG. 7, which provides a schematic longitudinal cross-sectional view of the exemplary embodiment of the elongated tubular body 76 as shown in FIG. 5.

Flexible zones 72, 73, 74, and 75 are, in this embodiment, implemented by providing the respective cylindrical elements with slits 72a, 73a, 74a, and 75a, respectively. Such slits 72a, 73a, 74a, and 75a may be arranged in any suitable pattern such that the flexible zones 72, 73, 74, and 75 have a desired flexibility in the longitudinal and tangential direction in accordance with a desired design.

FIG. 7 shows a longitudinal cross section of the four layers or cylindrical elements mentioned above, i.e. the inner cylindrical element 101, the first intermediate cylindrical element 102, the second intermediate cylindrical element 103, and the outer cylindrical element 104.

The inner cylindrical element 101, as seen along its length from the distal end to the proximal end of the instrument, comprises a rigid ring 111, which is arranged at the distal end part 13 of the steerable instrument 10, a first flexible portion 112, a first intermediate rigid portion 113, a second flexible portion 114, a second intermediate rigid portion 115, a third flexible portion 116, a third intermediate rigid portion 117, a fourth flexible portion 118, and a rigid end portion 119, which is arranged at the proximal end portion 11 of the steerable instrument.

The first intermediate cylindrical element 102, as seen along its length from the distal end to the proximal end of the instrument, comprises a rigid ring 121, a first flexible portion 122, a first intermediate rigid portion 123, a second flexible portion 124, a second intermediate rigid portion 125, a third flexible portion 126, a third intermediate rigid portion 127, a fourth flexible portion 128, and a rigid end portion 129. The portions 122, 123, 124, 125, 126, 127 and 128 together form a longitudinal steering wire 120 that can be moved in the longitudinal direction like a wire. The longitudinal dimensions of the rigid ring 121, the first flexible portion 122, the first intermediate rigid portion 123, the second flexible portion 124, the second intermediate rigid portion 125, the third flexible portion 126, the third intermediate rigid portion 127, the fourth flexible portion 128, and the rigid end portion 129 of the first intermediate element 102, respectively, are aligned with, and preferably approximately equal to the longitudinal dimensions of the rigid ring 111, the first flexible portion 112, the first intermediate rigid portion 113, the second flexible portion 114, the second intermediate rigid portion 115, the third flexible portion 116, the third intermediate rigid portion 117, the fourth flexible portion 118, and the rigid end portion 119 of the inner cylindrical element 101, respectively, and are coinciding with these portions as well. In this description “approximately equal” means that respective same dimensions are equal within a margin of less than 10%, preferably less than 5%.

Similarly, the first intermediate cylindrical element 102 comprises one or more other longitudinal steering wires of which one is shown with reference number 120a.

The second intermediate cylindrical element 103, as seen along its length from the distal end to the proximal end of the instrument, comprises a first rigid ring 131, a first flexible portion 132, a second rigid ring 133, a second flexible portion 134, a first intermediate rigid portion 135, a first intermediate flexible portion 136, a second intermediate rigid portion 137, a second intermediate flexible portion 138, and a rigid end portion 139. The portions 133, 134, 135 and 136 together form a longitudinal steering wire 130 that can be moved in the longitudinal direction like a wire. The longitudinal dimensions of the first rigid ring 131, the first flexible portion 132 together with the second rigid ring 133 and the second flexible portion 134, the first intermediate rigid portion 135, the first intermediate flexible portion 136, the second intermediate rigid portion 137, the second intermediate flexible portion 138, and the rigid end portion 139 of the second intermediate cylinder 103, respectively, are aligned with, and preferably approximately equal to the longitudinal dimensions of the rigid ring 111, the first flexible portion 112, the first intermediate rigid portion 113, the second flexible portion 114, the second intermediate rigid portion 115, the third flexible portion 116, the third intermediate rigid portion 117, the fourth flexible portion 118, and the rigid end portion 119 of the first intermediate element 102, respectively, and are coinciding with these portions as well.

Similarly, the second intermediate cylindrical element 103 comprises one or more other longitudinal steering wires of which one is shown with reference number 130a.

The outer cylindrical element 104, as seen along its length from the distal end to the proximal end of the instrument, comprises a first rigid ring 141, a first flexible portion 142, a first intermediate rigid portion 143, a second flexible portion 144, and a second rigid ring 145. The longitudinal dimensions of the first flexible portion 142, the first intermediate rigid portion 143 and the second flexible portion 144 of the outer cylindrical element 104, respectively, are aligned with, and preferably approximately equal to the longitudinal dimension of the second flexible portion 134, the first intermediate rigid portion 135 and the first intermediate flexible portion 136 of the second intermediate element 103, respectively, and are coinciding with these portions as well. The rigid ring 141 has approximately the same length as the rigid ring 133 and is fixedly attached thereto, e.g. by spot welding or gluing. Preferably, the rigid ring 145 overlaps with the second intermediate rigid portion 137 only over a length that is required to make an adequate fixed attachment between the rigid ring 145 and the second intermediate rigid portion 137, respectively, e.g. by spot welding or gluing. The rigid rings 111, 121 and 131 are attached to each other, e.g., by spot welding or gluing. This may be done at the end edges thereof but also at a distance of these end edges.

In an embodiment, the same may apply to the rigid end portions 119, 129 and 139, which can be attached to one another as well in a comparable manner. However, the construction may be such that the diameter of the cylindrical elements at the proximal portion is larger, or smaller, with respect to the diameter at the distal portion. In such embodiment the construction at the proximal portion differs from the one shown in FIG. 7. As a result of the increase or decrease in diameter an amplification or attenuation is achieved, i.e., the bending angle of a flexible zone at the distal portion will be larger or smaller than the bending angle of a corresponding flexible portion at the proximal portion.

The inner and outer diameters of the cylindrical elements 101, 102, 103, and 104 are chosen in such a way at a same location along the elongated tubular body 76 that the outer diameter of inner cylindrical element 101 is slightly less than the inner diameter of the first intermediate cylindrical element 102, the outer diameter of the first intermediate cylindrical element 102 is slightly less than the inner diameter of the second intermediate cylindrical element 103 and the outer diameter of the second intermediate cylindrical element 103 is slightly less than the inner diameter of the outer cylindrical element 104, in such a way that a sliding movement of the adjacent cylindrical elements with respect to each other is possible. The dimensioning should be such that a sliding fit is provided between adjacent elements. A clearance between adjacent elements may generally be in the order of 0.02 to 0.1 mm, but depends on the specific application and material used. The clearance may be smaller than a wall thickness of the longitudinal steering wires to prevent an overlapping configuration thereof. Restricting the clearance to about 30% to 40% of the wall thickness of the longitudinal steering wires is generally sufficient.

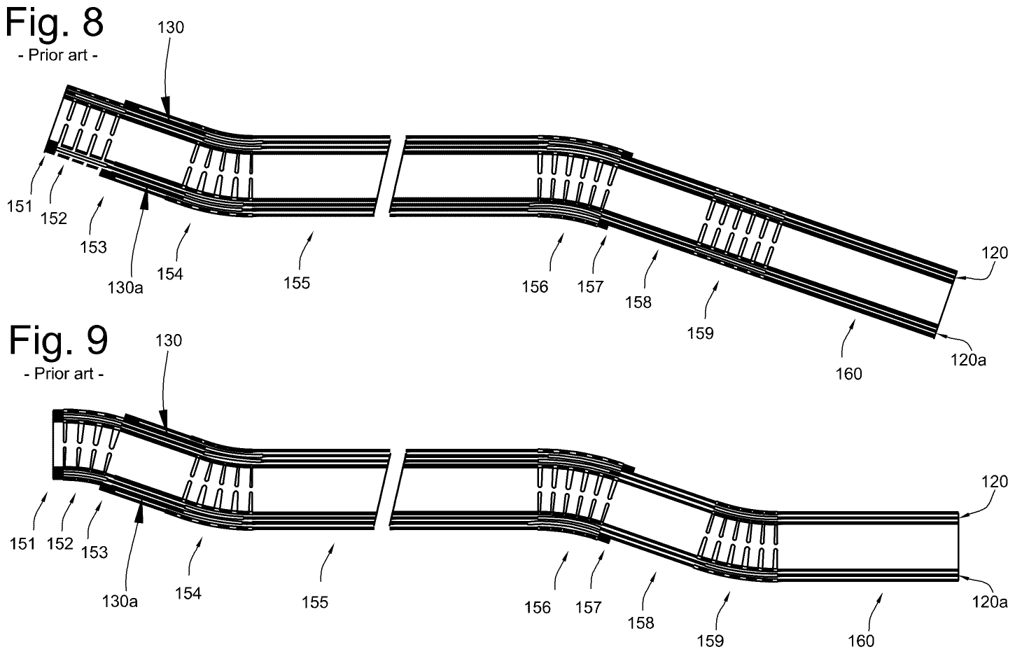

As can be seen in FIG. 7, flexible zone 72 of the proximal end part 11 is connected to the flexible zone 74 of the distal end part 13 by portions 134, 135 and 136, of the second intermediate cylindrical element 103, which form a first set of longitudinal steering wires of the steering arrangement of the steerable instrument. Furthermore, flexible zone 73 of the proximal end part 11 is connected to the flexible zone 75 of the distal end part 13 by portions 122, 123, 124, 125, 126, 127, and 128 of the first intermediate cylindrical element 102, which form a second set of longitudinal steering wires of the steering arrangement. The use of the construction as described above allows the steerable instrument 10 to be used for double bending. The working principle of this construction will be explained with respect to the examples shown in FIGS. 8 and 9.

For the sake of convenience, as shown in FIGS. 7, 8 and 9, the different portions of the cylindrical elements 101, 102, 103, and 104 have been grouped into zones 151-160 that are defined as follows. Zone 151 comprises the rigid rings 111, 121, and 131. Zone 152 comprises the portions 112, 122, and 132. Zone 153 comprises the rigid rings 133 and 141 and the portions 113 and 123. Zone 154 comprises the portions 114, 124, 134 and 142. Zone 155 comprises the portions 115, 125, 135 and 143. Zone 156 comprises the portions 116, 126, 136 and 144. Zone 157 comprises the rigid ring 145 and the parts of the portions 117, 127, and 137 coinciding therewith. Zone 158 comprises the parts of the portions 117, 127, and 137 outside zone 157. Zone 159 comprises the portions 118, 128 and 138. Finally, zone 160 comprises the rigid end portions 119, 129 and 139.

In order to deflect at least a part of the distal end part 13 of the steerable instrument, it is possible to apply a bending force, in any radial direction, to zone 158. According to the examples shown in FIGS. 8 and 9, zone 158 is bent downwards with respect to zone 155. Consequently, zone 156 is bent downwards. Because of the first set of longitudinal steering wires comprising portions 134, 135, and 136 of the second intermediate cylindrical element 103 that are arranged between the second intermediate rigid portion 137 and the second rigid ring 133, the downward bending of zone 156 is transferred by a longitudinal displacement of the first set of longitudinal steering wires into an upward bending of zone 154 with respect to zone 155. This is shown in both FIGS. 8 and 9.

It is to be noted that the exemplary downward bending of zone 156, only results in the upward bending of zone 154 at the distal end of the instrument as shown in FIG. 8. Bending of zone 152 as a result of the bending of zone 156 is prevented by zone 153 that is arranged between zones 152 and 154. When subsequently a bending force, in any radial direction, is applied to the zone 160, zone 159 is also bent. As shown in FIG. 9, zone 160 is bent in an upward direction with respect to its position shown in FIG. 8. Consequently, zone 159 is bent in an upward direction. Because of the second set of longitudinal steering wires comprising portions 122, 123, 124, 125, 126, 127 and 128 of the first intermediate cylindrical element 102 that are arranged between the rigid ring 121 and the rigid end portion 129, the upward bending of zone 159 is transferred by a longitudinal displacement of the second set of longitudinal steering wires into a downward bending of zone 152 with respect to its position shown in FIG. 8.

FIG. 9 further shows that the initial bending of the instrument in zone 154 as shown in FIG. 8 will be maintained because this bending is only governed by the bending of zone 156, whereas the bending of zone 152 is only governed by the bending of zone 159 as described above. Due to the fact that zones 152 and 154 are bendable independently with respect to each other, it is possible to give the distal end part 13 of the steerable instrument a position and longitudinal axis direction that are independent from each other. In particular the distal end part 13 can assume an advantageous S-like shape. The skilled person will appreciate that the capability to independently bend zones 152 and 154 with respect to each other, significantly enhances the maneuverability of the distal end part 13 and therefore of the steerable instrument as a whole.

Obviously, it is possible to vary the lengths of the flexible portions shown in FIGS. 7 to 9 as to accommodate specific requirements with regard to bending radii and total lengths of the distal end part 13 and the proximal end part 11 of the steerable instrument or to accommodate amplification or attenuation ratios between bending of at least a part of the proximal end part 11 and at least a part of the distal end part 13.

In the shown embodiment, the longitudinal steering wires comprise one or more sets of longitudinal steering wires that form integral parts of the one or more intermediate cylindrical elements 102, 103. Preferably, the longitudinal steering wires comprise remaining parts of the wall of an intermediate cylindrical element 102, 103 after the wall of the intermediate cylindrical element 102, 103 has been provided with longitudinal slits that define the remaining longitudinal steering wires.

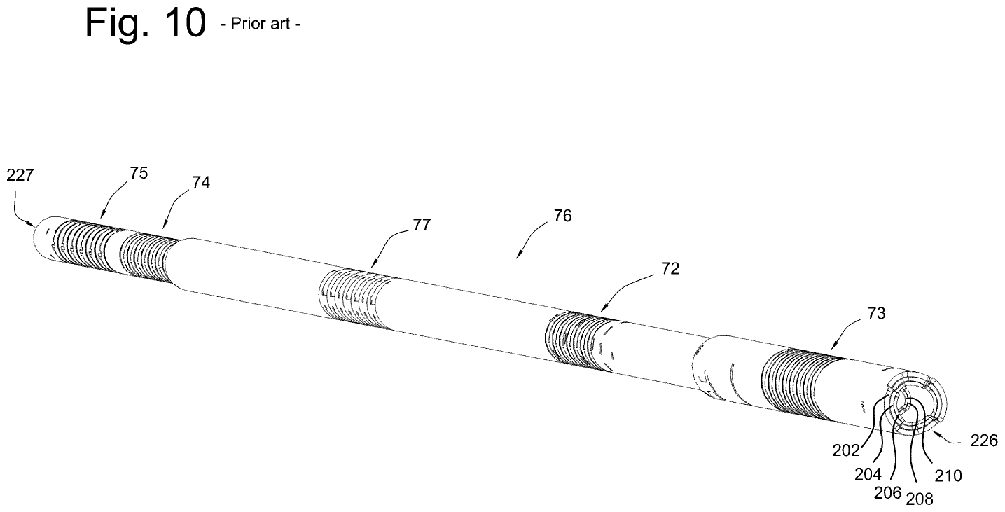

FIG. 10 shows a 3D view of an example of a steerable instrument. Like reference numbers refer to the same elements as in other figures. Their explanation is not repeated here. The instruments comprises five coaxial cylindrical elements 202-210. An inner cylindrical element 210 is surrounded by intermediate cylindrical element 208 which is surrounded by intermediate cylindrical element 206 which is surrounded by intermediate cylindrical element 204 which is, finally surrounded by outer cylindrical element 202. Inner intermediate cylindrical element may be made of a flexible spiraling spring. The proximal and distal ends, respectively, of the instrument are indicated with reference numbers 226 and 227, respectively.

As shown, here, instrument 76 comprises a flexible zone 77 in its intermediate part between flexible zone 72 and flexible zone 74. I.e., intermediate cylindrical element 204 (which is located at the outer side in the area of flexible zone 77) is provided with a slotted structure to provide intermediate cylindrical element with a desired flexibility. The longitudinal length of the slotted structure in flexible zone 77 depends on the desired application. It may be as long as the entire part between flexible zones 72 and 74. All other cylindrical elements 206, 208, 210 inside intermediate cylindrical element 204 are also flexible in flexible zone 77. Those cylindrical elements that have steering wires in the flexible zone 77 should be made as flexible as possible in that zone 77. Others are provided with suitable hinges, preferably made by suitable slotted structures.

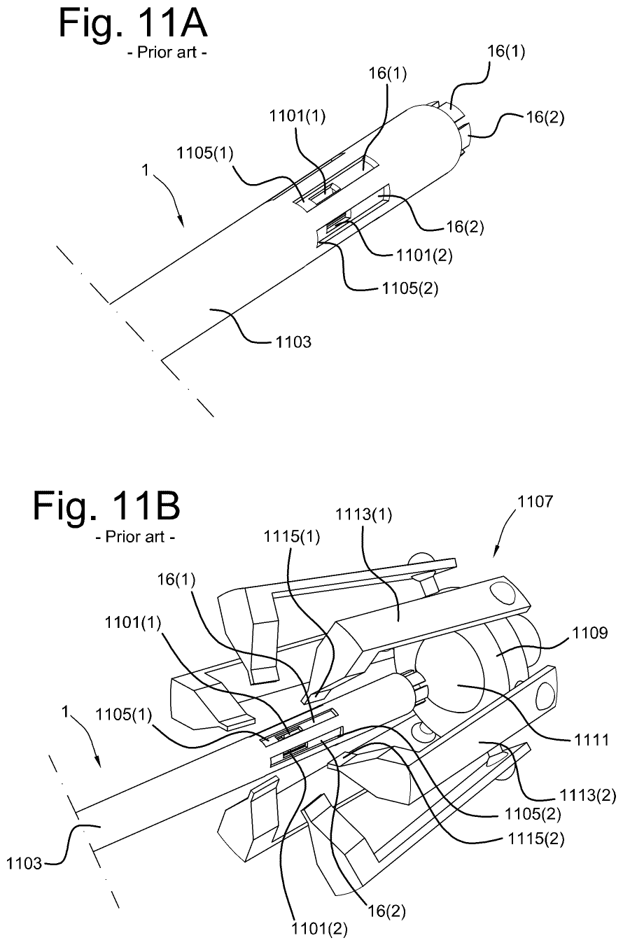

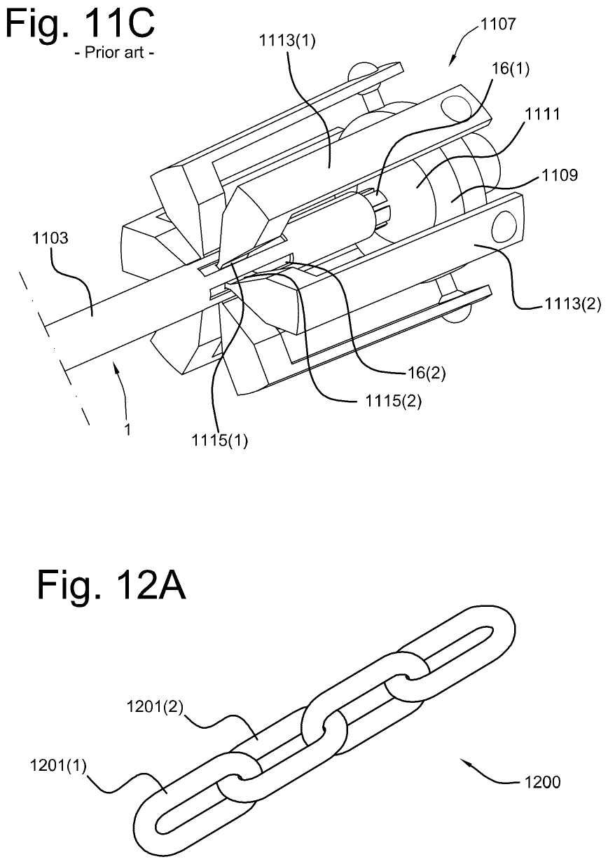

In FIGS. 1-10 the steering wires 16 are caused to move in the longitudinal direction by bending one or more bendable zones at the proximal end of the instrument. However, steering wires 16 can also be made to move in their longitudinal direction by simply moving them in that direction by other dives like robotic controlled devices. One example is given in FIGS. 11A-11C.

FIGS. 11A-11C show a prior art arrangement for controlling longitudinal movement of one or more steering wires 16(j) (j=1, 2, . . . , J) at the proximal end of the instrument, as is explained in WO2020218921A2.

The instrument 1 comprises an outer tube 1103 covering the steering wires 16(j). The outer tube 1103 comprises a plurality of openings 1105((j), i.e., one per steering wire 16(j). Each steering wire 16(j) of instrument 1 also comprise one or more openings 1101(j) overlapping with respective openings 1105(j) of outer tube 1103.

The openings 1105(j) of outer tube 1103 and the openings 1101(j) of the steering wires 16(j) may result from laser cutting in respective cylindrical tubes inserted into one another. As an alternative to laser cutting other techniques may be used, e.g., cutting by means of water jets. Also, other methods such as 3D laser printing may be used. These openings 1101(j) and 1105(j) extend through the whole thickness of the material.

FIG. 11B shows an example of the instrument 1 and a steering device 1107 wherein the steering device 1107 and the instrument 1 are detached. In the shown example, the steering device 1107 comprises a steering unit 1109 and a supporting unit 1111 wherein the steering unit 1109 is rotationally mounted on the supporting unit 1111. The steering unit 1109 comprises a plurality of arm-shaped elements 1113(j) fixedly connected to the steering unit 1109 and extending outwardly from the steering unit for connecting each one of the plurality of longitudinal elements 16(j) to one of the plurality of arm-shaped elements 1113(j) of the steering unit 1109.

FIG. 11C shows the instrument 1 and the steering device 1107 of FIG. 11B connected together by inserting one end part of a plurality of end parts 1115(j) of arm-shaped element 1113(j) into an opening 1101(j) of one steering wire 16(j) such that, by steering the steering unit 1109 around the supporting unit 1111, the arm-shaped elements 1113(j) may pull or push the steering wires 16(j) in the longitudinal direction of the instrument for controlling deflection of one or more deflectable zones 13, 152, 154 of the distal end of the instrument 1.

Such steering is accomplished by rotating steering unit 1309 in three dimensions about ball shaped supporting unit 1111. Control of such rotation may be implemented by a handle which can be manually controlled or controlled by a robotic device. A robotic device may also steer each steering wire individually, without the use of steering unit 1109.

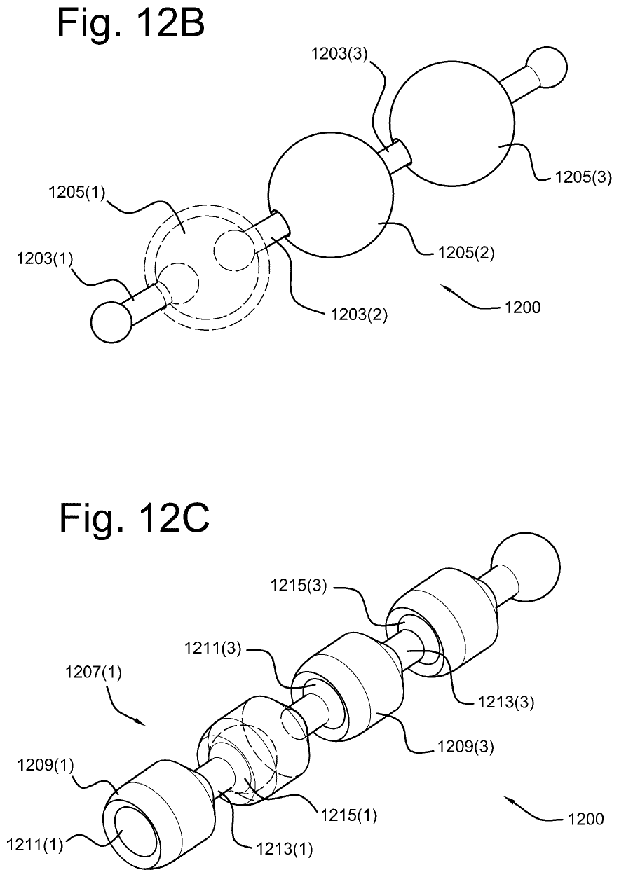

FIGS. 12A, 12B, and 12C show prior art shackled chain links. FIG. 12A shows a plurality of shackled chain links 1201(k) (k=1, 2, 3, . . . , K) in which each chain link has the form of a closed curved structure with an opening that may be circular, elliptical, or have any other suitable form. Each chain link 1201(k) passes through the opening of its adjacent chain links 1201(k) such as to form a chain.

FIG. 12B shows an arrangement with a plurality of hollow balls 1205(k) wherein adjacent hollow balls 1205(k), 1205(k+1) are connected by means of a rod 1203(k+1). Rod 1203(k+1) passes through suitable openings in adjacent hollow balls 1205(k), 1205(k+1) and is provided with an extension inside adjacent hollow balls 1205(k), 1205(k+1) to prevent rod 1203(k+1) to be retractable from hollow balls 1205(k), 1205(k+1) through these openings.

The chains of FIGS. 12A and 12B can withstand pull forces and do not have bending stiffness, but these chains cannot transfer push forces. This is different with the arrangement of FIG. 12C.

FIG. 12C shows a variant to the arrangement of FIG. 12B in which each chain link 1209(k) is provided with a ball shaped cavity 1211(k) at a first end and a rod 1213(k) at a second end opposing the first end. Each rod 1213(k) is provided with a ball shaped end portion 1215(k) having a size matching the size of cavity 1211(k+1) of adjacent chain link 1209(k+1) such that ball shaped end portion 1215(k) can freely rotate inside cavity 1213(k+1) but not be withdrawn from cavity 1213(k+1).

The variant of FIG. 12C the advantage that it can be made with the required strength and that it has no bending stiffness other than some friction forces between adjacent chain links 1209(k).

One known disadvantage of known steerable instruments is that they are often built from many separate parts and are difficult to assemble. Because of the usually complex structure, they also often require a high degree of maintenance and are prone to mechanical failure. These instruments and the use of these instruments generally are expensive and they have to be used many times to get costs per use down to commercially acceptable levels. Making steering elements from many separate parts of course would add to this complexity but still is a viable solution in some cases.

The prior art instrument as discussed with reference to FIGS. 1-10 addresses the need for a reliable and single use steerable instrument that can be manufactured and commercialized against acceptable costs. As will be explained in detail hereinafter, also in this type of instrument it is possible to apply shackled chain links in steering wires based on the principles shown in FIGS. 12A-12C. Such shackled chain links can also be made integrally with the tube and pre-assembled like the rest of the instrument layers in a material removal process, like laser cutting, water jet cutting, etching, Electro Discharge Machining (EDM) or chipping from a metal or plastic tube.

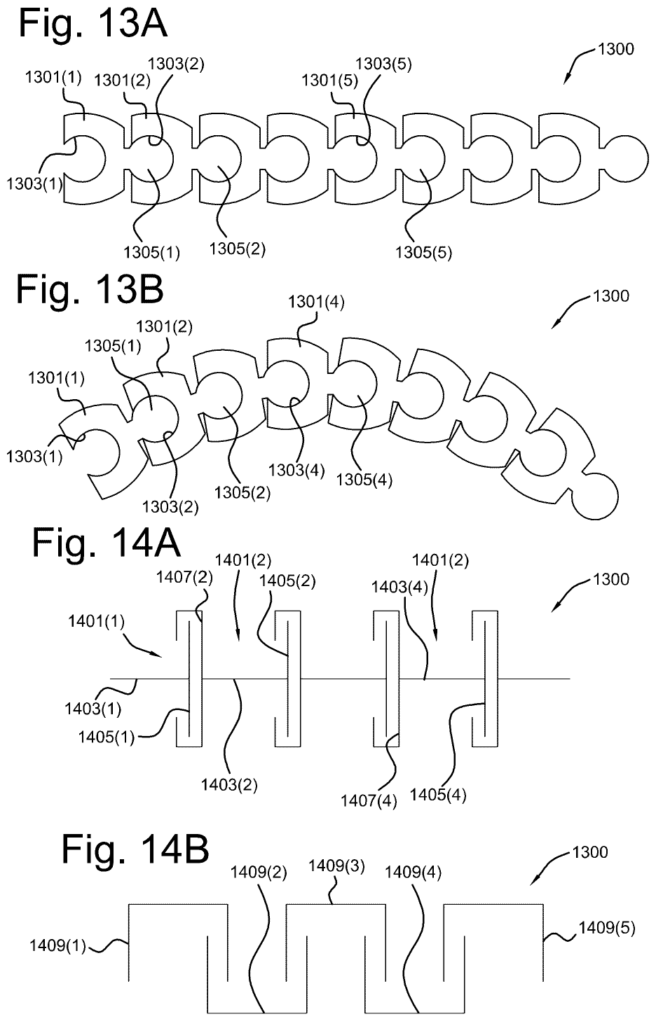

In one of its simplest forms a flexible steering wire part 1300 implemented by shackled chain links of a strip like steering wire 16(j) could look like the one shown in FIGS. 13A, 13B.

FIG. 13A shows flexible steering wire part 1300 having a series of adjacent chain links 1301(k). Each chain link 1301(k) is provided with a circular opening 1303(k) at a first end and a circular shaped extension 1305(k) at a second end opposing the first end. Each circular shaped extension 1305(k) is rotatably arranged inside circular opening 1303(k+1) of adjacent chain link 1301(k+1). To that end, circular shaped extension 1305(k) has a size matching a size of opening 1303(k+1). Moreover, opening 1303(k+1) extends along a circular arc of more than 180 degrees such that circular shaped extension 1305(k) cannot be retracted from opening 1303(k+1). As can be seen in FIG. 13A, the chain links only form connections with adjacent chain links along the longitudinal direction of the steering wire. The chain link structure can hence be seen as a free-standing structure, extending along the steering wire and separated from other parts of the tube and from any adjacent chain link structure in the circumferential direction. The chain link in fact forms part of the steering wire.

FIG. 13B shows the chain link structure of FIG. 13A once it is bent. As shown adjacent chain links 1301(k), 1301(k+1) can be shaped such that they can rotate relative to one another to a certain predetermined maximum angle in which they contact one another and block further rotation from circular shaped extension 1305(k) inside circular opening 1303(k+1). When implemented to form at least one steering wire 16(j) of a steerable instrument, e.g. to form one or more steering wires 16 as illustrated in FIGS. 2-10, the adjacent chain links forming the portion of the steering wire may rotate relative to one another in a tangential plane of the tube.

The structure of FIGS. 13A and 13B may be made by cutting suitable slot patterns in a tube. Then, its outside surface, as projected on the drawing surface, is curved in the circumferential direction of the tube but straight in its longitudinal direction. Its thickness is the same as the thickness of the tube from which it is made. Rotation of adjacent chain links 1301(k), 1301(k+1) relative to one another is then in a plane perpendicular to a radial direction which radial direction is defined as a direction perpendicular to a longitudinal axis of the instrument. That is, the adjacent chain links may rotate relative to one another in the tangential plane of the tube.

The embodiment of FIGS. 13A and 13B can be seen as an implementation of a basic shape shown in FIG. 14A. FIG. 14A shows a chain with a plurality of adjacent shackled chain links 1401(k). Each chain link 1401(k) has a rod or strip 1403(k) provided with an enlarged end portion 1405(k) extending into an opening 1407(k+1) such that enlarged end portion 1405(k) can rotate in opening 1407(k+1) in a tangential plane of the tube in which the chain is formed, and enlarged end portion 1405(k) cannot be retracted from opening 1407(k+1). In this way, adjacent chain links 1401(k), 1401(k+1) can rotate relative to one another in this tangential plane while, at the same time a longitudinal pulling/pushing force exerted on one of them is transferred to an identical longitudinal pulling/pushing force on the other one. The amount of play depends on the spaces between adjacent chain links 1401(k), 1401(k+1).

FIG. 14B shows another basic shape of a series of adjacent chain links 1409(k). In FIG. 14B, each chain link 1409(k) has a U-shaped form with a U-shaped opening. Adjacent chain links 1409(k), 1409(k+1) have respective U-shaped openings 180 rotated relative to one another as seen in a tangential plane, and hooking into one another. Other shapes, like a V-shape or horseshoe shape, may be applied as well.

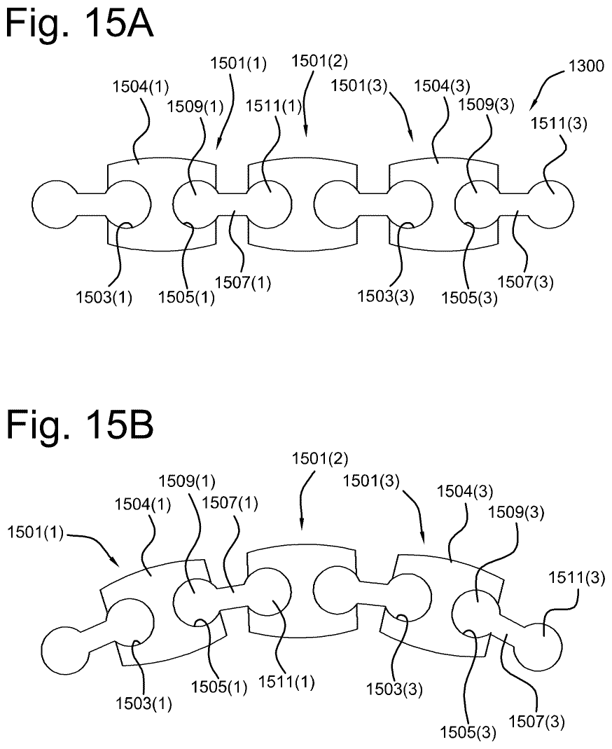

Some implementation examples of chain structures, which may be used to form a steering wire part in a flexible portion of an instrument, are explained with reference to FIGS. 15A, 15B, 16A, 16B, 17A, 17B hereinafter.

FIGS. 15A and 15B show an embodiment of flexible steering wire part 1300 in which adjacent chain links 1501(k) comprise two parts, i.e., a first chain link portion 1504(k) and a second chain link portion 1507(k). First chain link portion 1504(k) is provided with a first circular opening 1503(k) at one end and a second circular opening 1505(k) at a second end opposing the first end. Second chain link portion 1507(k) is a strip provided with circular end portions 1509(k), 1511(k) at both ends. One of these circular end portions 1509(k) is accommodated in second circular opening 1505(k). Circular end portion 1509(k) has a size which matches the size of second circular opening 1505(k) such that circular end portion 1509(k) can freely rotate inside second circular opening 1505(k). Moreover, second opening 1505(k) extends along a circular arc of more than 180 degrees such that circular end portion 1509(k) cannot be retracted from second opening 1505(k). Circular end portion 1509(k) and second circular opening 1505(k) are separated by a slot of which the width is determined by the used material removal technique. When a laser beam is used to produce the slot the slot width may be between 0.01 to 2.00 mm, more typically for this application, between 0.015 and 0.04 mm.

Circular end portion 1511(k) extends into a first opening 1503(k+1) of an adjacent chain link 1501(k+1). Circular end portion 1511(k) has a size which matches the size of first circular opening 1505(k+1) such that circular end portion 1511(k) can freely rotate inside first circular opening 1505(k+1). Moreover, first circular opening 1505(k+1) extends along a circular arc of more than 180 degrees such that circular end portion 1511(k) cannot be retracted from first circular opening 1505(k+1). Circular end portion 1511(k+1) and first circular opening 1505(k+1) are separated by a slot of which the width is determined by the used material removal technique. When a laser beam is used to produce the slot the slot width may be between 0.01 to 2.00 mm, more typically for this application, between 0.015 and 0.04 mm.

The flexible steering wire part 1300 of FIG. 15A is shown in a bent state in FIG. 15B. Herein, similar to described above with respect of FIGS. 13A and 13B, adjacent chain links 1501(k), 1501(k+1) rotate relative to one another in a tangential plane of the tube.

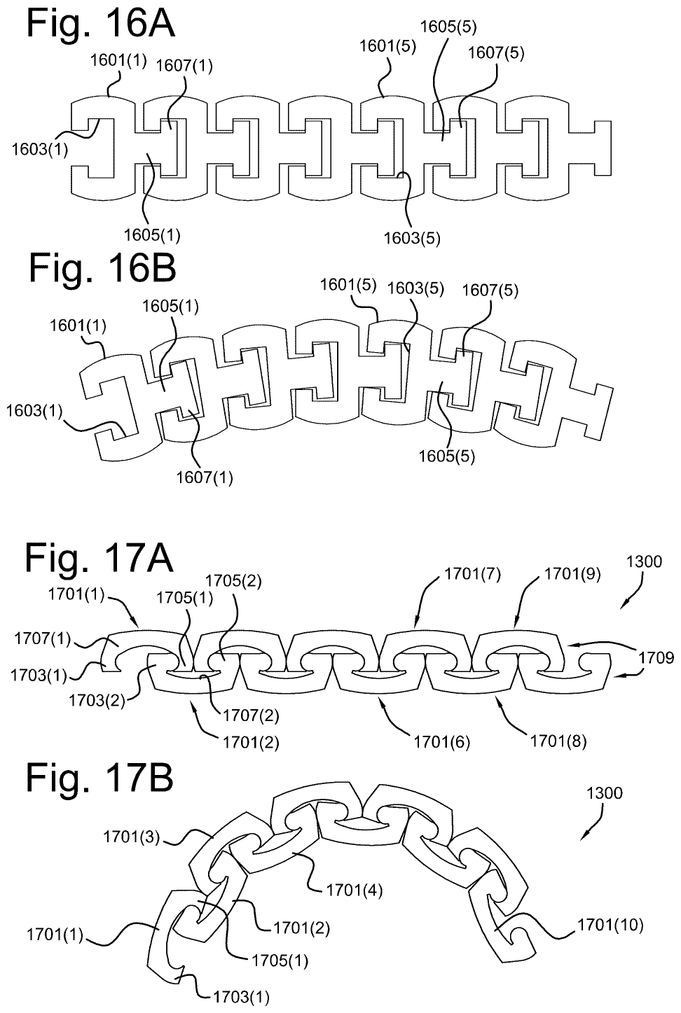

FIGS. 16A and 16B show a further embodiment of flexible steering wire part 1300 where all chain links 1601(k) are made as a single piece. Each chain link 1601(k) comprises an opening 1603(k) extending at a first end and a strip 1605(k) extending towards a second end opposite to the first end. Strip 1605(k) is provided with an enlarged end portion which, in the shown example, has the form of a transverse strip 1607(k) perpendicular to a longitudinal axis of strip 1605(k). Transverse strip portion 1607(k) is accommodated in opening 1603(k+1) of an adjacent chain link 1601(k+1) such that it cannot be retracted from it. However, in the shown embodiment, transverse strip portion 1607(k) can be longitudinally shifted inside opening 1603(k+1) with a certain predetermined play. Opening 1603(k+1) and transverse strip portion 1607(k) are dimensioned such that transverse strip 1607(k) can tilt inside opening 1605(k+1) resulting in a rotation between adjacent chain links 1601(k) and 1601(k+1), as shown in FIG. 16B.

The same principle as shown in FIGS. 15A and 15B can be applied to the embodiment of FIGS. 16A and 16B. I.e., strip 1605(k) can be provided with extensions like transverse strip portion 1607(k) at both ends, one of which extending inside opening 1503(k+1) and one extending in a similar opening inside a separate portion of chain link 1601(k), like portion 1504(k) (cf. FIGS. 15A and 15B).

FIGS. 17A and 17B show an embodiment of chain links 1701(k) in accordance with the basic principle of FIG. 14B.

Chain links 1701(k) have a horseshoe shape with end portions 1703(k), 1705(k) and an opening 1707(k). Horseshoe openings 1707(k), 1707(k+2), 1707(k+4), . . . face in one direction whereas horseshoe openings 1707(k+1), 1707(k+3), 1707(k+5), . . . face in an opposite direction such that horseshoe openings 1707(k), 1707(k+2), 1707(k+4), respectively, are interlocking with horseshoe openings 1707(k−1)/1707(k+1), 1707(k+1)/1707(k+3), and 1707(k+3)/1707(k+5), respectively. In the embodiment shown in FIGS. 17A and 17B there is hardly any play between adjacent chain links 1701(k), 1701(k+1). However, if desired some play may be implemented.

As illustrated in FIGS. 17A and 17B, lateral sides 1709 of the chain links 1701(k) in the longitudinal direction may be slanted or oblique, allowing adjacent chain links 1701(k), 1701(k+1) to rotate relative one another. These may rotate to a certain degree, set by the degree to which the sides are slanted or oblique, until portions of the sides of adjacent chain links abut one another, as shown in FIG. 17B. Thereby, adjacent chain links may rotate relative to one another in a tangential plane extending through the chain structure in a wall of a tube wherein the steering wire is formed, the maximum amount of bending being defined by the shape of the chain links.

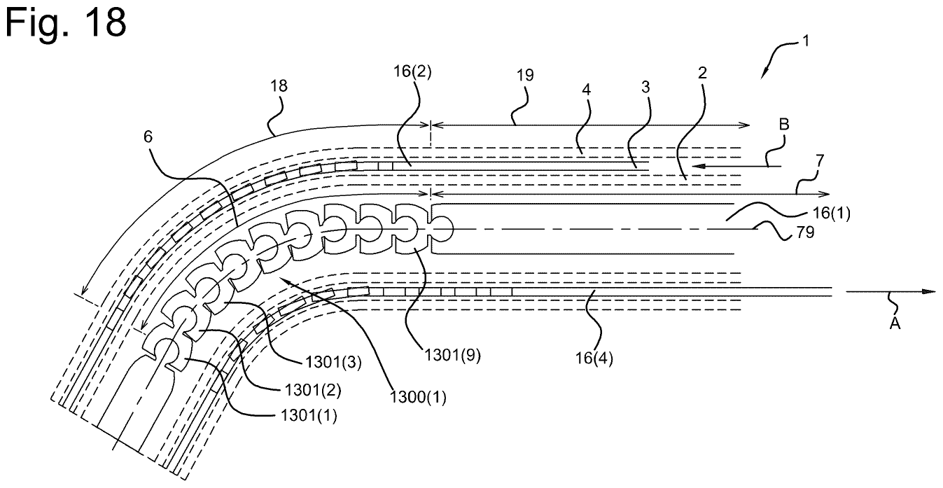

FIG. 18 shows a longitudinal cross section through steerable instrument 1 having steering wires 16(j) with a flexible steering part 1300 as shown in FIGS. 13A and 13B. The steerable instrument is an instrument as shown in FIGS. 1, 2 and 3 but its principle is equally well applicable in other steerable instruments like the ones shown in FIGS. 4-10.

Inner tube 2 and outer tube 4 are not shown in FIG. 18. Their location is only shown by means of dotted lines 2 and 4. Therefore, one can see the inside of steering wire 16(1) with flexible steering part 1300, provided in the wall of tube 3. Reference sign 79 refers to a central axis of steerable instrument 1. Moreover, one sees steering wires 16(2) and 16(4) longitudinally cut in half in this view.

As one can see, flexible steering part 1300 is in a bent state in FIG. 18 as caused by steering wire 16(4) being pulled in a proximal direction A and steering wire 16(2) being pushed in a distal direction B.

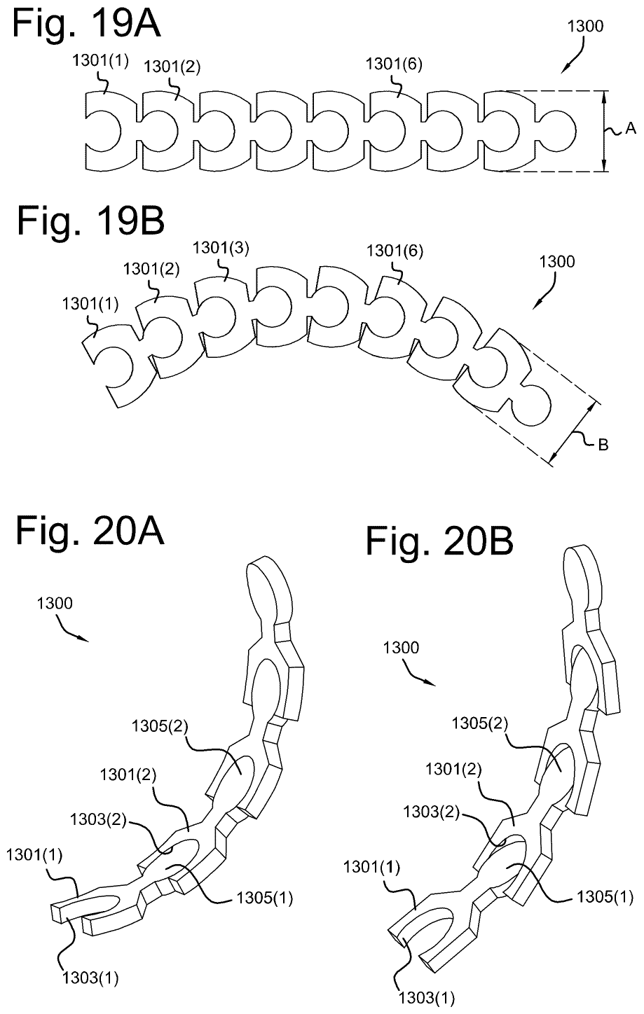

It is observed that all shown embodiments of chain links in FIGS. 13A, 13B, 15A, 15B, 16A, 16B, 17A, 17B and 18 are designed with such shapes that once a series of shackled chain links is in a bent position, the tangential width of that series remains the same. This is shown in FIGS. 19A and 19B. FIG. 19A shows the embodiment of FIGS. 13A and 13B having a tangential width A in an unbent condition and width B in a bent condition. Ideally, the outer shape of chain links 1301(k) is such that width A equals width B. That may be implemented by making that shape curved, e.g. like a portion of a circular arc.

A steering wire cut from a tube wall or a sheet, only has the ‘chain of shackles’ geometry in its cutting plane. However, in a steerable instrument a steering wire 16(j) has to be able to bend in all directions. That can be explained with reference to FIG. 18. In FIG. 18, steerable instrument 1 is bent such that flexible steering part 1300 in steering wire 16(1) is bent in a plane which is perpendicular to a perpendicular through central axis 79 of steerable instrument 1 and a central axis of flexible steering part 1300. However, as is also visible from FIG. 18, flexible steering parts 1300 of steering wires 16(2) and 16(4) are oriented tangentially 90 degrees rotated relative to steering wire 16(1). So, in the bent portion of steerable instrument 1, these flexible steering parts 1300 of steering wires 16(2) and 16(4) are bent in a direction perpendicular to the bending direction of flexible steering part 1300 of steering wire 16(1).

One could accept that the bending stiffness in that perpendicular bending direction is minimized by the chain geometry allowing adjacent chain links 1301(k) to rotate relative to one another and that the bending stiffness in the direction perpendicular to that bending direction is the same as for a standard, straight cut element that bends by elastic deformation. This is shown in FIG. 20A which shows a 3D schematic view of flexible steering part 1300 of e.g. steering wire 16(2) in the bending condition of FIG. 18. Here, the chain links 1301(k) bend in a direction perpendicular to their surface which results in some stiffness. So, in the bending condition of FIG. 18, flexible steering parts 1300 of steering wires 16(2) and 16(4) still provide some stiffness to the bending. However, flexible steering parts 1300 of steering wires 16(1) and 16(3) (not visible in FIG. 18) would have no bending stiffness resulting in an improved bending performance of steerable instrument 1.

In practise, when consecutive chain links 1301(k) are made from a tube with a material removal technique, as explained above, there is, however, a certain play between them because of the slots between them. Then, as shown in FIG. 20B, circular shaped extension 1305(k) of chain link 1301(k) can also rotate inside circular opening 1303(k+1) of an adjacent chain link in a plane perpendicular to the surface of the chain link 1301(k). This would reduce the total bending stiffness of a bendable zone of instrument 1 even more.

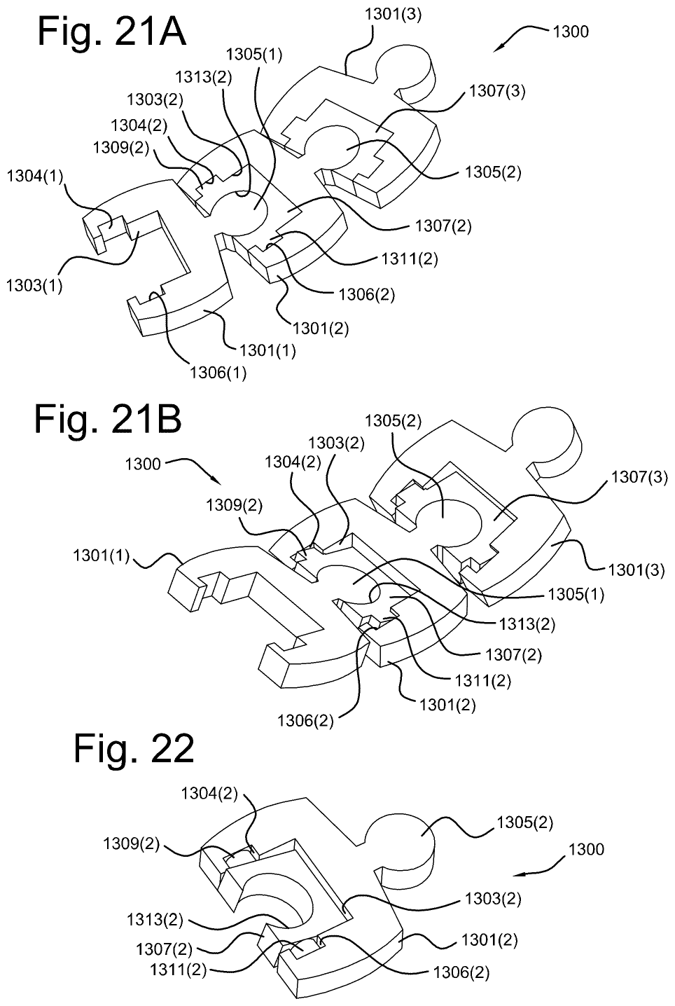

FIGS. 21A and 21B show an embodiment improving the possibility of bending consecutive chain links 1301(k) in the plane perpendicular to the plane of the chain links 1301(k) themselves. To that effect, chain link 1301(k) is provided with a rotatable portion 1307(k) located inside opening 1303(k). Rotatable portion 1307(k) is provided with two pins 1309(k), 1311(k) extending in opposite directions perpendicular to a central axis of chain link 1301(k). Moreover, rotatable portion 1307(k) is provided with a circular opening 1313(k) accommodating circular shaped extension 1305(k−1). Circular shaped extension 1305(k−1) has a size matching a size of opening 1313(k). Moreover, opening 1313(k) extends along a circular arc of more than 180 degrees such that circular shaped extension 1305(k−1) cannot be retracted from opening 1303(k).

The two pins 1309(k) and 1311(k) are located inside respective notches 1304(k) and 1306(k) in opening 1303(k) such that they can rotate inside respective notches 1304(k) and 1306(k) allowing adjacent chain links 1301(k−1) and 1301(k) to also rotate in the plane perpendicular to the surface of chain links 1301(k−1) and 1301(k).

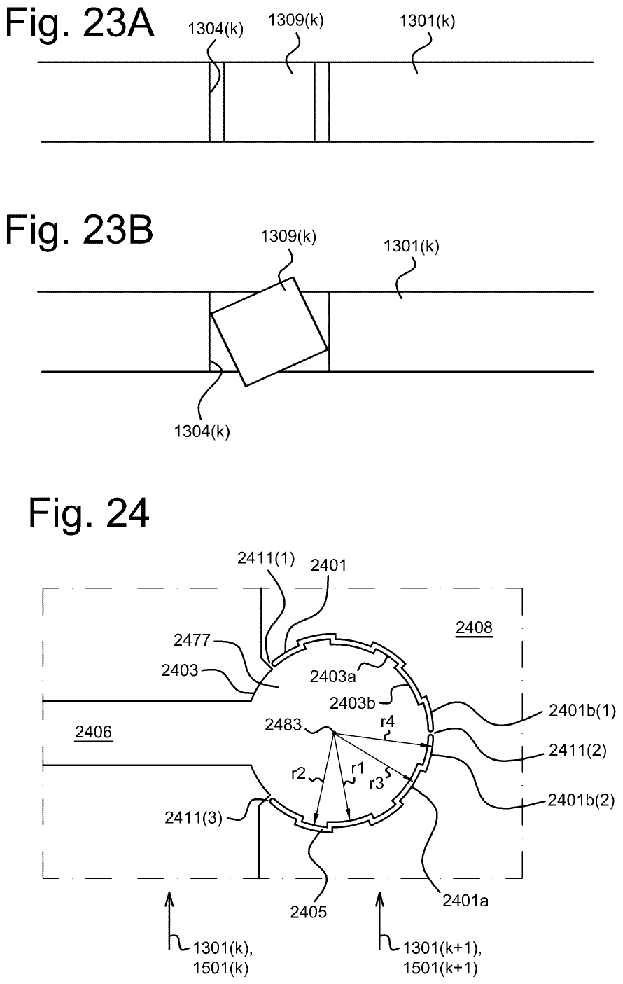

In the embodiment of FIGS. 21A and 21B, the distance between pins 1309(k) and 1311(k), respectively, and notches 1304(k) and 1306(k), respectively, is determined by the used material removing technique to produce the slots between them, cf. cross section of FIG. 23A. If the width of the slots is wide enough relative to the thickness of the chain links (which is the same as the thickness of the tube used to make the structure) then pins 1309(k) and 1311(k), respectively, can freely rotate inside notches 1304(k) and 1306(k), respectively. If not, rotation will be blocked at a certain rotation angle, as shown in cross section view of FIG. 23B.

Rotation in a plane perpendicular to the surface of the chain links 1301(k) may be improved by the embodiment shown in FIG. 22. Here, pins 1301309(k) and 1311(k) that reside in notches 1304(k) and 1306(k) have a cylindrical shape, which could be the result of a cutting process in which layers of material are only locally removed without fully shooting through the material.

Now the series of chain links 1301(k) can bend in all directions without bending stiffness. Many more geometries are possible with bending axes in two planes. Also asymmetric geometries in which the bending stiffness in one direction has a higher magnitude than in the other direction can be envisioned. In practice, the bending capacity preferably is symmetric and the bending capacity in the cutting plane of the tube is the same as the bending capacity perpendicular to that plane. One can also limit the bending capacity to a certain amount. In this way one can make sure that per chain link length only a certain maximum bending angle can be achieved. In this way one can assure that if one, for example, wants to bend the instrument tip with 30 degrees and one designs for example 6 hinge structures and 6 chain links 1301(k) in the curve length, one hinge structure and one chain link 1301(k) can have a limited bending capacity of 5 degrees maximum. In this way one can assure that the bent tip has a nice round bending behaviour without local bends bigger than 5 degrees. This can positively affect fatigue life of the hinges, chain links 1301(k) and steering wires 16(j) and can prevent high friction forces in steering wires 16(j) that one would have when an instrument tip bends sharply in one location.

As one can see, tubes are used to manufacture instrument 1. Its operative portions, like hinges and steering wires, are made by providing the tubes with suitable, predetermined slotted patterns. Especially if these slotted structures are large or if they enclose a portion which is entirely separate from the rest of the tube structure through the slotted structure, manufacturing of the instrument may be complex because these operative portions may no longer be positioned in the original cylindrical shape of the tube directly after the slot forming process. This could make inserting tubes into one another complex. As known from WO2016/089202 from the present applicant, this can be solved by applying fracture elements bridging the slots at predetermined locations and fracturing these fracture elements after tubes have been inserted into one another. Such fracture elements can also be applied in the instrument of the present document, as will be explained with reference to FIG. 24, which also shows an example of how play between adjacent chain links can be reduced.

First an embodiment of reduction of play between adjacent chain links will be described.

FIG. 24 schematically shows some portions of adjacent chain links, e.g., chain links 1301(k), 1501(k). The figure shows two opposing chain link portions 2408, 2477 of, e.g., chain links 1301(k)/1501(k), 1301(k+1)/1501(k+1) in which play is reduced. Chain link portion 2408 of chain link 1301(k+1)/1501(k+1) has a circular opening 2475 with a serrated outside edge 2401 extending about a center point 2483. Chain link 1301(k)/1501(k) comprises a strip 2406 attached to a circular extension 2477. Circular extension 2477 has a serrated outside edge 2403 also extending about center point 2483.

The serrated outside edge 2403 of circular extension 2477 has extending portions 2403a and an indented portion 2403b between each two adjacent extending portions 2403a. Both the extending portions 2403a and the indented portions 2403b may have a circular form extending along a circle about center point 2483. However, they may have any other suitable form. In the shown embodiment, the indented portions 2403b extend along a first circle having a first radius r1. The extending portions 2403a extend along a second circle having a second radius r2 which is larger than the first radius r1.

The serrated outside edge 2401 of circular opening 2475 has extending portions 2401a and an indented portion 2401b between each two adjacent extending portions 2401a. Both the extending portions 2401a and the indented portions 2401b may have a circular form extending along a circle about center point 2483. However, they may have any other suitable form. In the shown embodiment, the extending portions 2401a extend along a third circle having a third radius r3. The indented portions 2401b extend along a fourth circle having a fourth radius r4 which is larger than the third radius r3.

FIG. 24 shows the two adjacent chain links 1301(k)/1501(k), 1301(k+1)/1501(k+1) in its status directly after it has been manufactured and not yet used in any way. The serrated outside edge 2403 of circular extension 2477 and the serrated outside edge 2401 of the circular opening 2475 are separated from one another by a slot 2405 which results from (laser) cutting adjacent chain links 1301(k)/1501(k), 1301(k+1)/1501(k+1) from a tube. This slot 2405 may have a constant width along its entire length, e.g., for medical applications in a range between 0.01 to 2.00 mm, more typically for this application, between 0.015 and 0.04 mm.

In an embodiment, the second radius r2 is about equal to the third radius r3. I.e., they may be equal within manufacturing tolerances which may be less than 10%, preferably less than 5% of r2 or r3. In the shown embodiment, the second radius r2 is not larger than the third radius r3 because otherwise circular extension 2477 cannot rotate inside circular opening 2475. An alternative definition is that the extending portions 2403a have a height which is at maximum about equal to the width of slot 2405 (or distance) between an adjacent indented portion 2403b and an opposing extending portion 2401a, where “about equal” again refers to equal within manufacturing tolerances, i.e., that height and distance differ by 10% or less, alternatively 5% or less, or further alternatively 1% or less.

FIG. 24 shows circular extension 2477 and circular opening 2475 in the resting state when they have not been rotated relative to one another. In the shown embodiment, the second radius r2 and third radius r3 are about equal such that, when circular extension 2477 and circular opening 2475 rotate relative to one another, an extending portion 2403a of circular extension 2477 abuts an extending portion 2401a of circular opening 2475. In case circular extension 2477 has a plurality of extending portions 2403a distributed along its outer edge 2403 and circular opening 2475 has a plurality of extending portions 2401a distributed along its outer edge 2401, several of the extending portions 2403a may, then, abut several of the extending portions 2401a. Depending on the exact design, several of the extending portions 2403a may abut several of the extending portions 2401a along a circular arc of a degrees about center point 2483 where a may be >45 degrees but a may alternatively be >180 degrees (as in FIG. 24).

At locations where one or more extending portions 2403a of circular extension 2477 abut one or more extending portions 2401a of circular opening 2475 they cannot move any more towards one another in the radial direction as seen from center point 2483. So, in the rotated status, play between abutting extending portions 2403a and extending portions 2401a is removed. Depending on the design, play may have been removed in at least one of the longitudinal direction or tangential direction of the tube in which the chain links 1301(k)/1501(k), 1301(k+1)/1501(k+1) are made.

Outer edge 2403 of circular extension 2477 has a transition edge portion between each extending portion 2403a and each adjacent indented portion 2403b. Outer edge 2401 of circular opening 2475 has a transition edge portion between each extending portion 2401a and each adjacent indented portion 2401b. Transition edge portions of outer edge 2403 and transition edge portions of outer edge 2401 are separated from one another by a distance which, after manufacturing, is as wide as the width of slot 2405 resulting from the cutting process. The width of slot 2405 at locations between opposing transition edge portions of outer edge 2403 and transition edge portions of outer edge 2401 may be as wide as the width of slot 2405 at locations between other opposing portions of outer edges 2403 and outer edge 2401 but that is not necessary.

If the width of slot 2405 at locations between opposing transition edge portions of outer edge 2403 and transition edge portions of outer edge 2401 is very small relative to the radius of circular extension 2477, only a very small rotation between circular extension 2477 and circular opening 2475 will result in extending portions 2403a of circular extension 2477 abutting extending portions 2401a of circular opening 2475. Consequently, only when two adjacent chain links 1301(k)/1501(k), 1301(k+1)/1501(k+1) are not rotated relative to one another, i.e. the status equal to the resting status, they show some play relative to one another which is as large as the width of slot 2405. In a relative rotated (or deflected) status about a certain deflection angle β, however, all play may be removed. In a typical example, such deflection angle β may <5 degrees or even <3 degrees or <1 degrees. In use, many adjacent chain links 1301(k)/1501(k), 1301(k+1)/1501(k+1) of invasive instrument 1 may be bent relative to one another about an angle >β, e.g. due to curvatures in a canal, for instance a human intestinal canal, in which the instrument is inserted. So, in use, a high percentage of play between adjacent chain links 1301(k)/1501(k), 1301(k+1)/1501(k+1) may be reduced by the embodiment of FIG. 24.

It is observed that the slots resulting from the manufacturing process to make the different operative parts of instrument 1 cause some play between such operative parts. Such play can be minimized with similar techniques as explained with reference to FIG. 24. Reference is made to not pre-published Dutch patent application NL2028739 which describes such techniques in detail.

Now a possible use of fracture elements between adjacent chain links 1301(k)/1501(k), 1301(k+1)/1501(k+1) is described.

As shown in FIG. 24, the adjacent chain links 1301(k)/1501(k), 1301(k+1)/1501(k+1) are still attached to one another by means of one or more fracture elements 2411(m) (m=1, 2, . . . , M). Such fracture elements 2411(m) keep circular extension 2477 of chain link 1301(k)/1501(k) and opposing chain link portion 2408 of chain link 1301(k+1)/1501(k+1) together during further assembly of the instrument, e.g., when one tube is inserted into another one. In use, i.e., when a predetermined minimum force is applied to rotate adjacent chain links 1301(k)/1501(k), 1301(k+1)/1501(k+1) relative to one another these fracture elements 2411(m) will fracture and play no role anymore. In the example of FIG. 24, three such fracture elements 2411(m) are provided. Of course, there can be less or more such fracture elements 2411(m).

Here, fracture element 2411(m) is shown to be a bridge between circular extension 2477 and opposing chain link portion 2408. However, fracture element 2411(m) can have any suitable design, as explained in above mentioned patent application WO2016/089202 of the present applicant.

In general, as e.g. explained herein below with reference to fracture elements 2411(m), such fracture elements can be designed in the following way. Fracture elements are made in the same process step as other elements are cut from a tube. Before being fractured, each fracture element is attached to opposite portions of two tube portions. In this way they keep these two opposite portions together and prevent the two portions from falling apart after the cutting process. These opposite portions have a geometrical shape such that the stresses in the fracture element will increase more than the stresses in the surrounding material and/or structure during manipulation. Therefore, if a deflection or a high enough force is applied on the two opposite portions such as to try to move them relative to one another, the stress in the fracture element rises above the yield stress of the tube material, causing permanent deflection of the fracture element. Applying even more deflection or a higher force results in the stress reaching the ultimate tensile stress of the fracture element causing a fracture of fracture element without causing a permanent deformation of the two portions because the stress as developed in the two portions remains below their yield stress.

In this way elements of the tubes that should be independently movable relative to one another in the final steerable instrument can be separated by operating the instrument after the different tubes are inserted into one another and the elements cannot fall apart anymore. I.e., the process of fracturing is preferably done when the steerable instrument is finished and all tubes are inserted into one another, and the elements that should be attached to one another have been attached.

Fracture elements 2411(m) should be designed in the following way. Before being fractured, each fracture element 2411(m) is attached to opposite portions of the tube from which the chain links are made. These opposite portions of the tube have a geometrical shape such that the stresses in the fracture element 2411(m) are higher than in the surrounding material and/or structure. Therefore, if a deflection or a high enough force is applied on a structure with a fracture element 2411(m)—here caused by rotating adjacent chain links relative to one another—the stress in the fracture element rises above the yield stress of the tube material, causing permanent deflection of fracture element 2411(m). Applying even more deflection or a higher force results in the stress reaching the ultimate tensile stress causing a fracture of fracture element 2411(m). An other mechanism to break the fracture element may be achieved by applying low or high cycle fatigue to fracture element 2411(m). The stress in fracture element 2411(m) is raised above the fatigue limit, causing a fatigue fracture when a sufficient number of deflections is used. In all cases the stresses in the surrounding structure/material stays at least below the yield stress of the tube material.

Fracture elements as described herein above may be applied to any adjacent elements of the embodiments of the present application in analogous manner.

Concepts of using fracture elements are schematically shown in FIGS. 31, 32 and 33. FIG. 31 shows a fracture element 4306 attached to a first tube portion 4302 and a second tube portion 4304 of a tube 4300. Here, fracture element 4306 has the form of a small disk attached to first and second tube portions 4302, 4304 via small bridges. In FIG. 31 first and second tube portions 4302 and 4304 can move relative to one another in the longitudinal direction of the tube 4300. The bridges of fracture element 4306 to the opposite first and second portions 4302 and 4304 will fracture once they move relative to one another and the above stress conditions apply.

FIG. 32 shows an embodiment with two opposite first and second tube portions 4302, 4304 which, during assembly, remain attached to one another by fracture element 4306 in the form of a small bridge. In this embodiment, opposite first and second tube element 4302, 4304 can rotate relative to one another in the surface of the drawing as indicated with arrow 4402. Once they rotate relative to one another forces are developed inside fracture element 4306 and inside the surrounding material of the opposite tube elements 4302, 4304 until a moment fracture element 4306 fractures because the stress inside fracture element 4306 rises above the ultimate tensile stress, as explained above.

FIG. 33 shows an alternative to the one shown in FIG. 44 in which first and second tube portions 4302, 4304 can rotate relative to one another as indicated with an arrow 4502. Now, fracture element 4306 has the shape of a small disk attached to the two opposite tube portions 4302, 4304 by means of small bridges. In this embodiment these bridges will fracture under the above explained stress conditions.

Although FIGS. 31, 32 and 33 show the application of fracture elements 4306 between two opposite tube portions 4302, 4304 that can move in the longitudinal direction relative to one another or rotate relative to one another, they can be used everywhere in the steerable instrument between two opposite tube portions that move relative to one another in usage of the steerable instrument, be it rotational, longitudinal, radial or tangential, because a large enough movement during use will eventually fracture these fracture elements 4306.

An other mechanism to break the fracture element 4306 may be achieved by applying low or high cycle fatigue to a fracture element. The stress in fracture element is raised above the fatigue limit, causing a fatigue fracture. Note that this fatigue limit is lower than the above mentioned ultimate tensile stress. In all cases the stresses in the surrounding structure/material of the two opposite tube elements to which fracture element 4306 is attached stays at least below the yield stress of the tube material. The process of fracturing by applying several fatigue cycles is preferably done when the steerable instrument is finished and all tubes are inserted into one another, and the elements that should be attached to one another have been attached.

As a further alternative, a fracture element may be melted by an energy, e.g. laser, beam after tubes are inserted into one another. Such an energy beam may be directed to the fracture element through a suitable hole in a tube outside the tube in which the fracture element is located. Of course, any combination of fracturing, applying fatigue and melting may be used if desired.

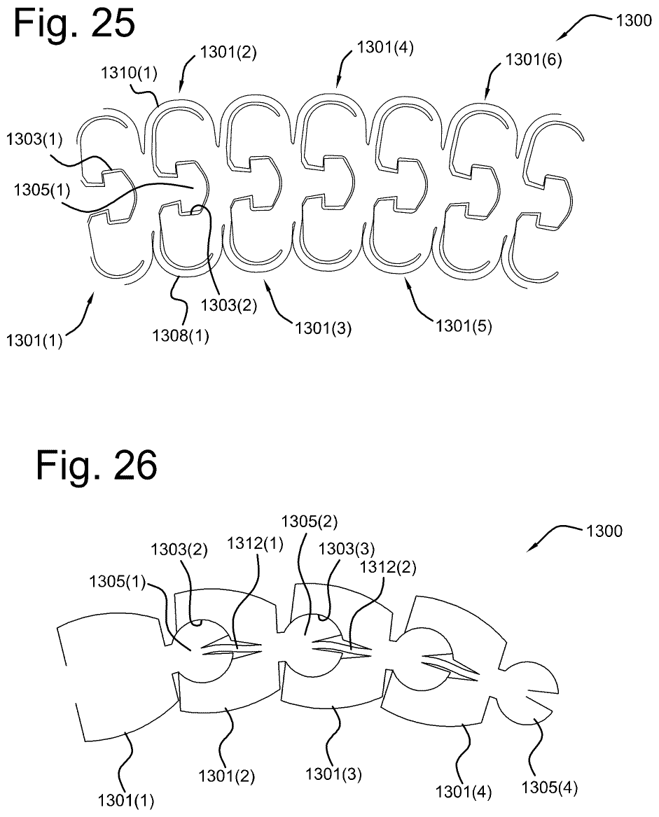

FIGS. 25 and 26 show embodiments of an alternative to using fracture elements. To avoid that after making slots by for example laser cutting, the structure of loose chain links falls apart before assembly into or onto the instrument, one could add small elastic bridges between the chain links of the steering wire.

FIG. 25 shows an example in which chain link 1301(k) is provided with one or more elastic bridge 1308(k), 1310(k) being at one end attached to an outer surface of chain link 1301(k) and at their other end to an outer surface of adjacent chain link 1301(k+1). In the example of FIG. 26, chain link 1301(k) is provided with one or more elastic bridges 1312(k) having one end attached to the extension 1305(k) of chain link 1301(k), which is here circular shaped and extending into opening 1303(k+1) of adjacent chain link 1301(k+1), and its other end attached to chain link 1301(k+1). The attachments at both ends are configured such that extension 1305(k) can still rotate inside opening 1303(k+1) to a certain predetermined amount.

When elastic bridges 1308(k), 1310(k), 1312(k) are used, the shackled chain links 1301(k) of steering wire 16(j) will gain a certain amount of bending stiffness, but when the elastic bridges 1308(k), 1310(k), 1312(k) have small dimensions as compared to what the width and thickness of a solid steering wire 16(j) would have, this increase of bending stiffness is minimal or even negligible.

Even though FIGS. 25 and 26 show the application of elastic bridges in a specific embodiment, they can be applied in any other embodiment as well.

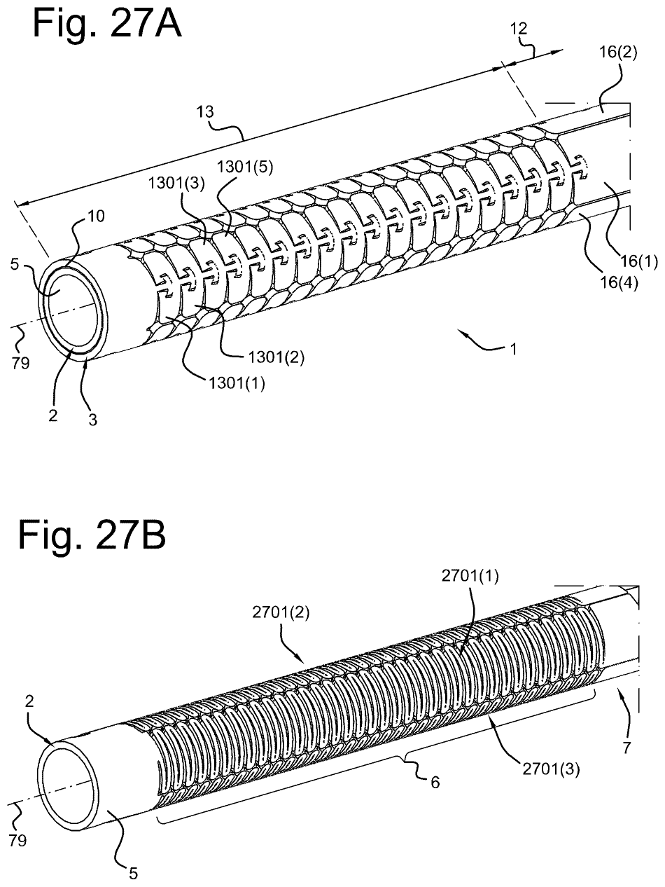

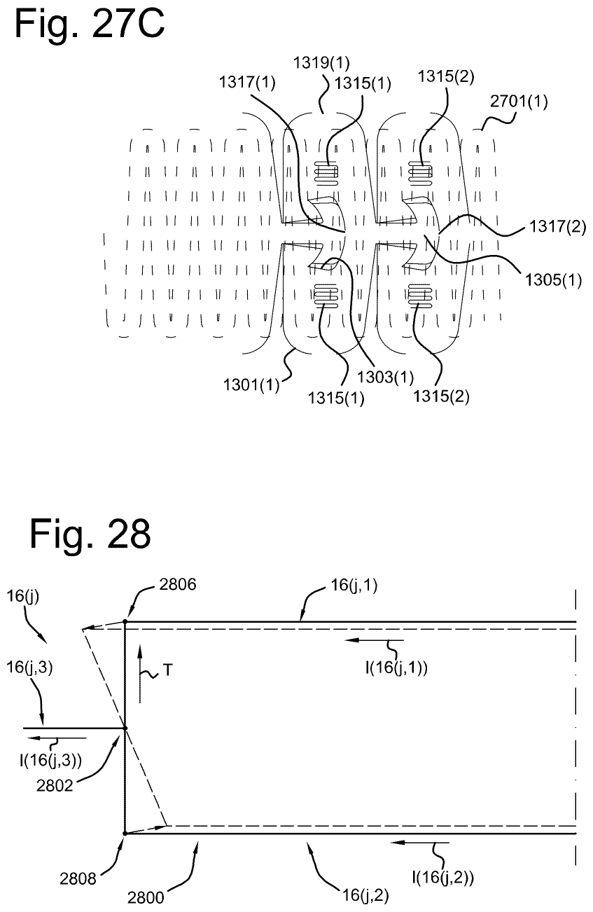

The invention can also be applied in multi-tube instruments in which portions of adjacent chain links manufactured from one tube are attached to portions of an adjacent tube, as will be explained with reference to FIGS. 27A, 27B, and 27C.

FIG. 27A show a two tube instrument 1 with inner tube 2 and intermediate tube 3 (cf. FIGS. 1, 2, and 3). Here the implementation of tubes 2 and 3 differs from the one shown in FIGS. 1, 2 and 3. I.e., in the distal end part 13 the flexible portion of steering wires 16(j) is implemented by a shackle of chain links according to the present invention, e.g. chain links 1301(k). in the intermediate part 12 the steering wires 16(j) are implemented as rather rigid strips separated from adjacent steering wires 16(j−1), 16(j+1) by a small slot resulting from the cutting process for instance, in a range between 0.01 to 2.00 mm, more typically for this application, between 0.015 and 0.04 mm.

As shown in the enlarged view of FIG. 27C, the extension of chain link 1301(k) extending into opening 1303(k+1) of chain link 1301(k+1) may have a different form than circular. Here, extension 1305(k) is shown to be a portion of a circular strip, whereas opening 1305(k+1) is shown to have a portion with the form circular slot. Circular strip extension 1305(k) and circular slot 1303(k+1) are configured such that circular strip extension 1305(k) can move inside circular slot 1303(k+1) such that adjacent chain links 1301(k), 1301(k+1) can rotate relative to one another.

Now, flexible bridges are made in an underlying tube layer. FIG. 27B shows that first flexible part 6 of inner tube 2 is implemented by a plurality of flexible wave form strips 2701(j), e.g. a sine wave or saw tooth form, one for each steering wire 16(j). Each wave form strip 2701(j) is both longitudinally and tangentially aligned with one series of chain links 1301(k) of steering wire 16(j) in distal end part 13.

FIG. 27C shows some adjacent chain links 1301(k), 1301(k+1) on top of wave form strip 2701(j) which is shown in dashed lines only. Every chain link 1301(k) is provided with one or more welding portions 1315(k), here made by providing chain link 1301(k) with one or more slots through the entire wall of the tube material. Such welding portions 1315(k) are welded, e.g. by laser welding, to a portion of wave form strip 2701(j) in the inner tube 2. By doing so, adjacent chain links 1301(k) and 1301(k+1) are now connected by an elastic member as in FIG. 25, but now the elastic member is made in a different tube layer as the chain links. By doing so, relative rotation between adjacent chain links 1301(k) and 1301(k+1) becomes slightly stiffer, but this effect is not very substantial, At the same time, however, via these attachments between chain links 1301(k) and wave form strip 2701(j) an elastic force is exerted on the chain links 1301(k) causing them to remain at their respective original radial and longitudinal locations as much as possible.

Instead of (laser) welding any other suitable attachment technique, like gluing, may be used.

Reference numbers 1317(k) and 1319(k), respectively, refer to fracture elements used to keep extension 1305(k) and other portions of chain link 1301(k) attached to adjacent parts of the tube during the process of cutting the slot pattern in the tube, which fracture elements are removed once the tube is inserted into another tube, as explained above.

It is observed that the arrangement shown in FIGS. 27A-27C are but one possible implementation to cause this effect.

As mentioned above, the clearance between adjacent tubes may generally be in the order of 0.02 to 0.1 mm, but depends on the specific application and material used. In order for adjacent chain links or force equalizing structure elements to fall apart in use of the instrument, the clearance is, preferably, smaller than a wall thickness of the tubes. Restricting the clearance to a value between 5-50%, preferably, between 30-40% of the wall thickness of the tubes is generally sufficient.

### Force Equalizer

In the above specification, adjacent portions of a steering wire connected to one another by means of a rotatable connection have been shown and explained in detail. A series of such adjacent portions of a steering wire may form chain links of a chain which provide the steering wire with a very large flexibility which can be advantageously used in flexible zones of the instrument where the steering wire should be highly flexible but still have a large strength in the longitudinal direction.

An alternative implementation of a portion of a steering wire 16(j) with a large flexibility and still large longitudinal strength will be explained with reference to FIGS. 28, 29B, 29A and 30. This implementation is based on the principle of a force equalizer (or “spreader bar device”). It is observed that applying the principle of a force equalizer in a steerable instrument in which the steering wires are produced as strips cut from a tube is known from FIGS. 12, 13 and 14 of WO2018/067004 from the present applicant.

To that end, first the principle of a force equalizer will be briefly explained with reference to FIG. 28. FIG. 28 shows a schematic view of a force equalizer structure 2800 implemented by a steering wire 16(j) which, along a certain portion of its length, is split in first and second adjacent steering wire portions 16(j,1), 16(j,2) which are both attached/connected to a third steering wire portion 16(j,3) of the steering wire 16(j). By splitting steering wire 16(j) in two adjacent steering wire portions 16(j,1), 16(j,2), steering wire 16(j) becomes more flexible in the zone in which these steering wire portions 16(j,1), 16(j,2) are located while keeping its total longitudinal strength. A problem, however, is that if steering wire 16(j) is bent in that zone in the plane of the drawing of FIG. 28—i.e., a plane perpendicular to a radius of the tube—the two steering wire portions 16(j,1), 16(j,2) will move in the longitudinal direction with different path lengths. This makes the point of transition between third steering wire portion 16(j,3) and the two steering wire portions 16(j,1), 16(j,2) vulnerable for deformation, force/friction increase and possible even rupture. This is solved by designing this point of transition as a force equalizer in which longitudinal path length differences or offsets in the two adjacent steering wire portions 16(j,1), 16(j,2) are equalized.

Third steering wire portion 16(j,3) is shown to have an end portion which is connected at a third point of connection 2802 to a transverse structure 2804 which, in rest, extends in a direction T perpendicular to the longitudinal direction of steering wire 16(j), as well as perpendicular to the radius of the tube. First steering wire portion 16(j,1) is connected to transverse structure 2804 at a first point of connection 2806. Second steering wire portion 16(j,2) is connected to transverse structure 2804 at a second point of connection 2808. In the preferred embodiment, third point of connection 2802 is located exactly between the first and second points of connection 2806 and 2808. It is to be understood that “exactly” means as close as possible to a location exactly in the middle between the other two points of connection in dependence on the used method of manufacturing. To guarantee that first and second steering wire portions 16(j,1), 16(j,2) are more flexible in the transverse direction than third steering wire portion 16(j,3) of steering wire 16(j) they are smaller in the transverse direction than the width of third steering wire portion 16(j,3).

The longitudinal movement of third steering wire portion 16(j,3), first steering wire portion 16(j,1) and second steering wire portion 16(j,2), respectively, is indicated with |(16(j,3)), |(16(j,1)) and |(16(j,2)), respectively. The direction in which the transverse structure 2804 extends when |(16(j,1))=|(16(j,2)) is indicated with a solid line in FIG. 28. The dashed line shows a direction of transverse structure 2804 once |(16(j,1)) and |(16(j,2)) are unequal, e.g., due to bending of them in the plane of FIG. 28.

If |(16(j,1)) and |(16(j,2)) are unequal, then transverse structure 2804 rotates relative to the third steering wire portion 16(j,3) about third point of connection 2802, rotates relative to first steering wire portion 16(j,1) about first point of connection 2806, and rotates relative to second steering wire portion 16(j,2) about second point of connection 2808. In this way, path length differences between first steering wire portion 16(j,1) and second steering wire portion 16(j,2) translate into rotations at three points 2802, 2806, 2808 of steering wire 16(j). As is evident to persons skilled in the art, even if first steering wire portion 16(j,1) and second steering wire portion 16(j,2) show a different path length movement, a longitudinal movement of third steering wire portion 16(j,3) translates into a longitudinal movement of both first steering wire portion 16(j,1) and second steering wire portion 16(j,2) (which movements may be slightly different).

In FIGS. 12, 13 and 14 of WO2018/067004 these three connection points 2802, 2806, 2808 are implemented with solid portions of steering wire 16(j) that are allowed to bend in order to allow the above mentioned rotations. However, such bending causes stresses in the material that might be a cause for early failure of this mechanisms due to fatigue issues. Furthermore, the required bending force to bend these elements adds stiffness to the steerable tip section of the instrument. The present document describes some improved examples with points of connection with free rotation allowing for larger path offsets.

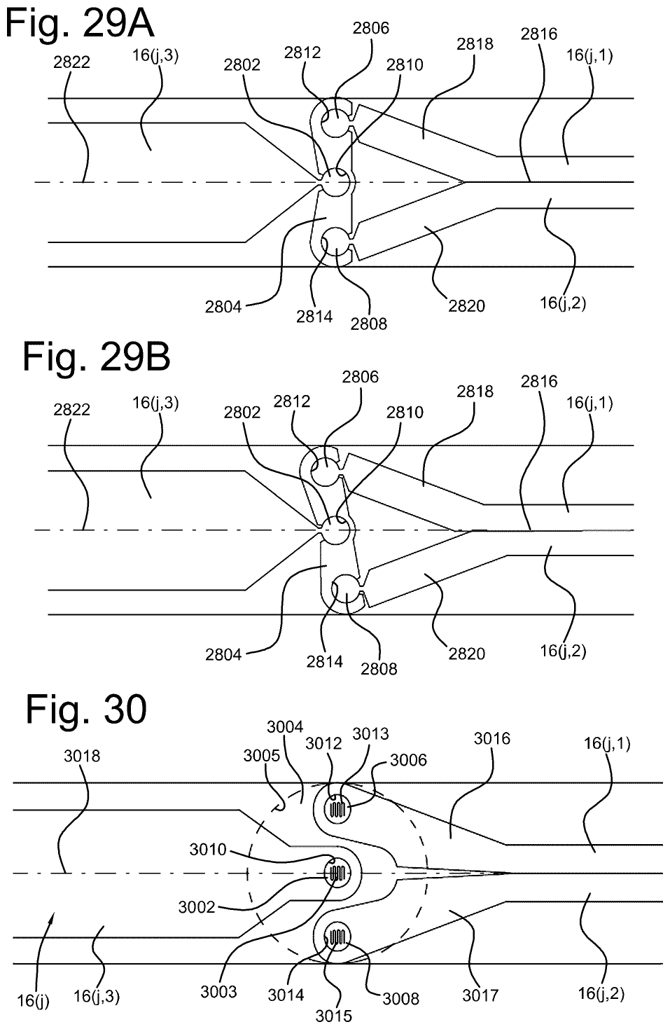

It is observed that in FIGS. 29A and 29B, third steering wire portion 16(j,3) has a larger width than first and second steering wire portions 16(j,1), 16(j,2). However, the principle of a force equalizer may equally well be applied in a situation where third steering wire portion 16(j,3) is substituted by a portion of steering wire 16(j) having a smaller width than first steering wire portion 16(j,1) and second steering wire portion 16(j,2).

FIGS. 29A and 29B show an implementation in which the points of connection 2802, 2806, 2808 are implemented by respective circular portions which are rotatably arranged inside a corresponding circular opening. I.e., in the shown embodiment, the end portion of the third steering wire portion 16(j,3) is provided with a third circular portion 2802 rotatably arranged inside a third circular opening 2810 inside transverse structure 2804 which is, here, implemented by a transverse strip 2804. Third circular opening 2810 extends along a portion of a circular arc larger than 180 degrees such that third circular portion 2802 cannot be retracted from third circular opening 2810.

At its opposite outer ends, transverse strip 2804 is provided with a first circular opening 2812 and a second circular opening 2814, respectively. First steering wire portion 16(j,1) is provided with a first end portion 2818 extending at a first angle of, e.g., between 5 and 40 degrees, preferably between 10 and 30 degrees, more preferably between 15 and 25 degrees relative to an axis of symmetry 2822 of steering wire 16(j) to first circular opening 2812. First end portion 2818 is provided with a first circular portion 2806 accommodated inside first circular opening 2812. Second steering wire portion 16(j,2) is provided with a second end portion 2820 extending at a second angle of, e.g., between 5 and 40 degrees, preferably between 10 and 30 degrees, more preferably between 15 and 25 degrees relative to axis of symmetry 2822 of steering wire 16(j) to second circular opening 2812. First and second angles may have the same value being it in opposite directions. Second end portion 2820 is provided with a second circular portion 2808 accommodated inside second circular opening 2814. All elements shown in FIGS. 29A, 29B are portions of the tube in which steering wire 16(j) is made and manufactured by providing the tube with a suitable slotted pattern.

First circular portion 2806, second circular portion 2808 and third circular portion 2802, respectively, have a play inside first circular opening 2812, second circular opening 2814 and third circular opening 2810, respectively, that may be between 0.01 to 2.00 mm, more typically for medical applications, between 0.015 and 0.04 mm.

FIG. 29A shows the structure in rest, i.e., a situation in which first and second steering wire portions 16(j,1), 16(j,2) have not moved relative to one another and in which, therefor, transverse strip is oriented perpendicular to the longitudinal direction of the tube and instrument.

FIG. 29B shows the same instrument in which first and second steering wire portions 16(j,1), 16(j,2) have moved relative to one another, e.g., caused by bending the instrument in the plane of the drawing in the zoned in which first and second steering wire portions 16(j,1), 16(j,2) are located. As one can see, in FIG. 29B, all three circular 2802, 2806 and 2808, respectively, are rotated inside openings 2810, 2812 and 2814, respectively, relative to the situation of FIG. 29A. These rotations do not cause any tension in the material of the transverse strip 2804 or anyone of first steering wire portion 16(j,3), second steering wire portion 16(j,2) and third steering wire portion 16(j,3), respectively, because the circular portions 2802, 2806 and 2808 can rotate freely inside openings 2810, 2812 and 2814, respectively. In this way, transverse strip 2804 operates as a spreader beam equalizing longitudinal force differences inside first and second steering wire portions 16(j,1) and 16(j,2).

At their opposing longitudinal ends, first and second steering wire portions 16(j,1), 16(j,2) may be attached to a still further steering wire portion (or to a fixed tube portion) by a further, similar force equalizer structure as the one shown in FIGS. 29A, 29B.

In order for the circular portions 2802, 2806 and 2808 to remain inside their respective openings 2810, 2812 and 2814 and not to fall out of them, the tube in which steering wire 16(j) is made should be inserted between an outer tube and inner tube. Moreover, the play between this tube and such inner tube and outer tube is less than the thickness of these tubes, e.g., the play may be in a range between 1-50% of this thickness

It is observed that FIGS. 29A, 29B show an embodiment in which the circular portions 2802, 2806 and 2808, respectively, are provided as portions of steering wire portions 16(j,3), 16(j,1) and 16(j,2), respectively, and all openings 2810, 2812 and 2814, respectively, are provided in transverse structure 2804. As one will understand, this may be reversed, i.e., the circular portions may be portions of transverse structure 2804 and the corresponding circular openings may be made in the respective steering wire portions 16(j,3), 16(j,1) and 16(j,2).

Moreover, it is observed that the circular portions 2802, 2806 and 2808 can be substituted by portions having another shape. Equally, the circular openings 2810, 2812, 2814 need not be fully circular. The only requirement is that these portions 2802, 2806, 2808 and openings 2810, 2812, 2814 are shaped such that these portions 2802, 2806, 2808 can rotate inside openings 2810, 2812, 2814, respectively, to a certain predetermined angle.

Fracture elements as explained with reference to FIG. 24 and described in detail herein above, can be applied as well in the embodiment of FIG. 29A, 29B.

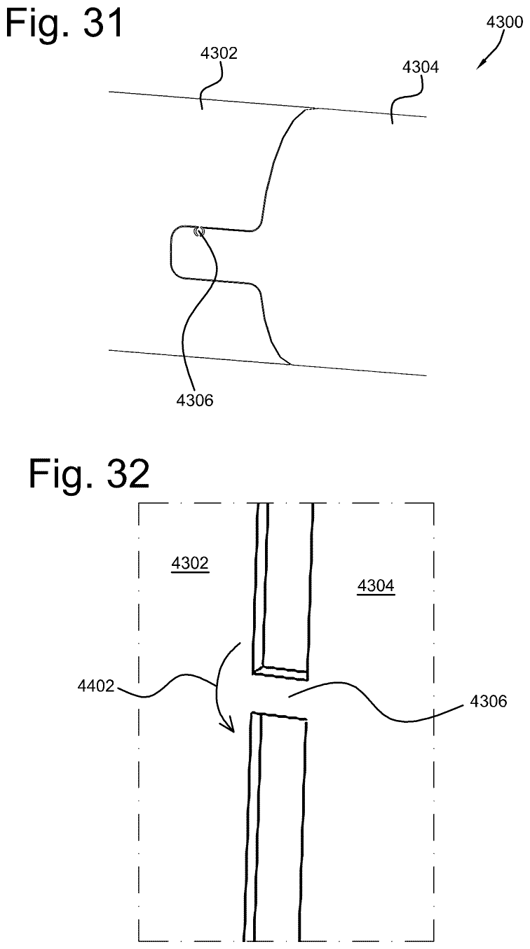

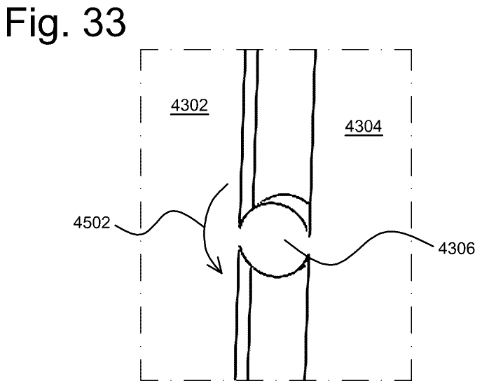

FIG. 30 shows a variant to the embodiment of FIGS. 29A, 29B. In FIG. 30, first steering wire portion 16(j,1) is provided with a first end portion 3016 facing third steering wire portion 16(j,3) and extending under a first angle away from an axis of symmetry 3018, which angle may be, e.g., between 5 and 40 degrees, preferably between 10 and 30 degrees, more preferably between 15 and 25 degrees. At its end, first end portion 3016 is provided with a first circular opening 3012 accommodating a first circular disk 3006.

Second steering wire portion 16(j,2) is provided with a second end portion 3017 facing third steering wire portion 16(j,3) and extending under a second angle away from an axis of symmetry 3018 in a direction opposite to the direction of first end portion 3016. The second angle is preferably the same as the first angle and may be, e.g., between 5 and 40 degrees, preferably between 10 and 30 degrees, more preferably between 15 and 25 degrees. At its end, second end portion 3017 is provided with a second circular opening 3014 accommodating a second circular disk 3008.

Preferably, a gap is provided between first end portion 3016 and second end portion 3017 which may be widening towards their ends (here, the distal ends of them), as shown in FIG. 30.

Third steering wire portion 16(j,3) is, at a third end portion facing first and second steering wire portions 16(j,1), 16(j,2), provided with a circular opening 3010 accommodating a circular disk 3002.

First circular disk 3006, second circular disk 3008 and third circular disk 3002, respectively, are, in the shown embodiment, provided with a first attachment structure 3013, a second attachment structure 3015 and a third attachment structure 3003, respectively, They may result from providing first circular disk 306, second circular disk 3008 and third circular disk 3002, respectively, with a suitable slotted pattern, e.g. a zig-zag pattern. The first attachment structure 3013, second attachment structure 3015 and third attachment structure 3003 may be used in a (laser) welding process to attach them to a rotatable structure 3004 located inside or outside the tube in which steering wire 16(j) is made.

Once third circular disk 3002 is attached to rotatable structure 3004 rotatable structure 3004 can rotate together with third circular disk 3002. Rotatable structure 3004 may have the form of a circular disk rotatably arranged inside a circular opening 3005 in the tube inside or outside the tube in which steering wire 16(j) is made. However, rotatable structure 3004 may have any other suitable form configured to rotate together with third circular disk 3002.

First circular disk 3006 is also attached to rotatable structure 3004 at a distance from circular disk 3002, preferably located at a line intersecting the center of rotatable disk 3002 and extending perpendicular to axis of symmetry 3018. Second circular disk 3008 is also attached to rotatable structure 3004 at a distance from circular disk 3002, also preferably located at that line but at a location opposite to the location of first circular disk 3006.

First circular disk 3006, second circular disk 3008 and third circular disk 3002, respectively, have a play inside first circular opening 3012, second circular opening 3014 and third circular opening 3010, respectively, that may be between 0.01 to 2.00 mm, more typically for medical applications, between 0.015 and 0.04 mm.

Third steering wire portion 16(j,3) is shown to have a larger width than first and second steering wire portions 16(j,1), 16(j,2), which may have the same width. In some applications, however, third steering wire portion 16(j,3) may have a width smaller than the width of first and second steering wire portions 16(j,1), 16(j,2).

In use, first and second steering wire portions, may be located in a zone of the instrument which is bendable. Like the embodiment of FIGS. 29A, 29B, then, first and second steering wire portions 16(j,1), 16(j,2) may bend in a plane of the drawing of FIG. 30, i.e., perpendicular to a radius of the tube in which steering wire 16(j) is made. Then, as explained above, first and second steering wire portions 16(j,1), 16(j,2) may move in the longitudinal direction of the instrument along different path lengths. In the embodiment of FIG. 30, this will result in third circular disk 3002 rotating about its own centre inside circular opening 3010, and first and second circular disks 3006, 3008 rotating in the same direction about third circular disk 3002 and inside their respective circular openings 3012, 3014. All these rotations have minimum friction.

Thus, the embodiment of FIG. 30 compensates path length differences between first and second steering wire portions 16(j,1), 16(j,2) in a similar way as the embodiment of FIGS. 29A, 29B. Once first and second steering wire portions 16(j,1), 16(j,2) move along different path lengths, first and second circular disks 3006, 3008 will rotate and their both longitudinal positions and tangential positions will change. I.e., as seen in the tangential direction they come closer to one another. Yet, first and second steering wire portions 16(j,1), 16(j,2) are prevented from being pressed against each other due to this tangential movement because of the gap between them.

As is evident to persons skilled in the art, even if first steering wire portion 16(j,1) and second steering wire portion 16(j,2) show a different path length movement, a longitudinal movement of third steering wire portion 16(j,3) translates into a longitudinal movement of both first steering wire portion 16(j,1) and second steering wire portion 16(j,2) (which movements may be slightly different). Next to that, a steering force that is applied to 16(j,3) is divided over 16(j,1) and 16(j,2) in a ratio equal to the perpendicular distance between the central axis through 16(j,3) and the centre of rotation of 3002 and 3006 respectively. If this distance is equal, the steering force F is divided in ½ F in wire 16(j,1) and ½ F in wire 16(j,2)

At their opposing longitudinal ends, first and second steering wire portions 16(j,1), 16(j,2) may be attached to a still further steering wire portion (or to a fixed tube portion) by a further, similar force equalizer structure as the one shown in FIG. 30.

In the embodiment shown in FIG. 30, the circular portions 3002, 3006 and 3008, respectively, may result from (laser) cutting them in respective steering wire portions 16(j,3), 16(j,1) and 16(j,2).

It is observed that FIG. 30 shows an embodiment in which the circular portions 3002, 3006 and 3008, respectively, are provided as portions of steering wire portions 16(j,3), 16(j,1) and 16(j,2), respectively, and all openings 3010, 3012 and 3014, respectively, are provided in portions of steering wire portions 16(j,3), 16(j,1) and 16(j,2) too. As one will understand, this may be reversed, i.e., the circular portions 3002, 3006 and 3008 and the corresponding circular openings 3010, 3012, 3014 may be portions of rotatable structure 3004 which are, then, attached to the respective steering wire portions 16(j,3), 16(j,1) and 16(j,2), e.g., by (laser) welding. In this alternative embodiment, the circular portions 3002, 3006 and 3008, respectively, may result from (laser) cutting them in rotatable structure 3004.

Moreover, it is observed that the circular portions 3002, 3006 and 3008 can be substituted by portions having another shape. Equally, the circular openings 3010, 3012, 3014 need not be fully circular. The only requirement is that these portions 3002, 3006, 3008 and openings 3010, 3012, 3014 are shaped such that these portions 3002, 3006, 3008 can rotate inside openings 3010, 3012, 3014, respectively, to a certain predetermined angle.

Fracture elements as explained with reference to FIG. 24 and described in detail herein above, can be applied as well in the embodiment of FIG. 30.

In order for the circular portions 3002, 3006 and 3008 to remain inside their respective openings 3010, 3012 and 3014 and not to fall out of them, the clearance between the tube in which steering wire 16(j) is made and the tube in which rotatable structure 3004 is made is, in an embodiment, less than the thickness of these tubes, e.g., the clearance may be in a range between 1-50%. Alternatively, the method used to attach the circular portions 3002, 3006 and 3008 to the adjacent tube may result in some extra material (e.g. glue or some welding material) being present in the space between them and the adjacent tube.

The thickness of cylindrical elements according to the described embodiments depend on their application. For medical applications the thickness may be in a range of 0.03-2.0 mm, preferably 0.03-1.0 mm, more preferably 0.05-0.5 mm, and most preferably 0.08-0.4 mm. The diameter of cylindrical elements depend on their application. For medical applications the diameter may be in a range of 0.5-20 mm, preferably 0.5-10 mm, more preferably 0.5-6 mm.

Longitudinal elements in one cylindrical element can be attached to longitudinal elements in adjacent cylindrical elements such that they are together operable to transfer a longitudinal motion from a longitudinal element at the proximal end of the instrument to a bendable portion of the instrument at the distal end of the instrument such that the bendable portion bends. This is explained in detail in WO 2017/213491 (cf. e.g. FIGS. 12, 13a and 13b in that PCT application) of the present applicant.

It will be clear to a person skilled in the art that the scope of the invention is not limited to the examples discussed in the foregoing, but that several amendments and modifications thereof are possible without deviating from the scope of the invention as defined in the attached claims. While the invention has been illustrated and described in detail in the figures and the description, such illustration and description are to be considered illustrative or exemplary only, and not restrictive. The present invention is not limited to the disclosed embodiments but comprises any combination of the disclosed embodiments that can come to an advantage.

Variations to the disclosed embodiments can be understood and effected by a person skilled in the art in practicing the claimed invention, from a study of the figures, the description and the attached claims. In the description and claims, the word “comprising” does not exclude other elements, and the indefinite article “a” or “an” does not exclude a plurality. In fact it is to be construed as meaning “at least one”. The mere fact that certain features are recited in mutually different dependent claims does not indicate that a combination of these features cannot be used to advantage. Any reference signs in the claims should not be construed as limiting the scope of the invention. Features of the above described embodiments and aspects can be combined unless their combining results in evident technical conflicts.

1. A steerable instrument comprising at least one tube extending in a longitudinal direction, the steerable instrument having a proximal end and a deflectable distal end, the at least one tube comprising at least one steering wire which is made from the at least one tube, separated by a slotted structure from the remainder of the at least one tube, attached to the deflectable distal end and configured to be movable in a longitudinal direction of the at least one tube such as to deflect the deflectable distal end, the at least one steering wire having at least one flexible portion located in a flexible zone of the steerable instrument and implemented by a series of adjacent chain links.

2. The steerable instrument according to claim 1, wherein adjacent chain links are configured to be rotatable in a first plane perpendicular to a radial direction which is defined as a direction perpendicular to a central axis of the steerable instrument, and, optionally, also rotatable in a second plane perpendicular to the first plane.

3. The steerable instrument according to claim 2, wherein the adjacent chain links are rotatable, within the first plane, relative to one another to a predetermined maximum angle in which they contact one another and block further rotation in the first plane.

4. The steerable instrument according to claim 1, wherein a bending capacity of the flexible portion of the steering wire is limited to a certain amount, preferably wherein the chain links are configured to define a maximum bending angle per chain link length.

5. The steerable instrument according to claim 1, wherein a first chain link is provided with an extension extending into an opening of an adjacent second chain link.

6. The steerable instrument according to claim 5, wherein the extension and the opening are shaped such that the extension cannot be retracted from the opening.

7. The steerable instrument according to claim 5, wherein both the extension and the opening have a circular shape.

8. The steerable instrument according to claim 6, wherein the extension and the second chain link are separated by a slot having a width in a range between 0.01 to 2.00 mm.

9. The steerable instrument according to claim 5, wherein the extension is an either straight or curved transverse strip.

10. The steerable instrument according to claim 5, wherein at least one chain link comprises a first chain link portion and a second chain link portion, the second chain link portion comprising a first extension extending into the opening as well as a second extension, the first chain link portion comprising a first opening connecting to an adjacent chain link and a second opening accommodating the second extension such that the first chain link portion and the second chain link portion can rotate relative to one another.

11. The steerable instrument according to claim 1, wherein chain links are shaped in one of a U-form, V-form, or horseshoe form.

12. The steerable instrument according to claim 1, wherein at least the first chain link is provided with a third chain link portion rotatably mounted inside the first chain link in a third plane perpendicular to the first plane.

13. The steerable instrument according to claim 1, wherein adjacent chain links are attached to one another by one or more flexible bridges.

14. The steerable instrument according to claim 1, wherein the steerable instrument comprises a further tube inside or outside the at least one tube, the further tube comprising a hinge structure in the flexible zone, one or more of the chain links being attached to the hinge structure.

15. The steerable instrument according to claim 14, wherein the hinge structure comprises a flexible wave form strip, e.g. a sine wave or saw tooth form, for each steering wire.

16. The steerable instrument according to claim 1, wherein the tube has a thickness in a range of 0.03-2.0 mm.

17. The steerable instrument according to claim 1, wherein the tube has a diameter in a range of 0.5-20 mm.

18.-27. (canceled)

28. A method of manufacturing a steerable instrument comprising at least one tube extending in a longitudinal direction, the steerable instrument having a proximal end and a deflectable distal end, the method comprising:
providing the at least one tube;
making at least one steering wire from the at least one tube, wherein the steering wire is attached to the distal end of said tube, by forming a slotted structure in a wall of the tube, thereby separating the steering wire from the remainder of the tube, and
providing the steering wire with a flexible portion located in a flexible zone of the steerable instrument, wherein the flexible portion is implemented by forming a series of adjacent chain links in at least a portion of the steering wire.

29. The method according to claim 28, wherein the at least one steering wire, including the series of adjacent chain links, is formed by cutting predetermined patterns in the tube.

30. The method according to claim 28, wherein the adjacent chain links are formed by a materials removal technique applied to the wall of the tube.

31. The method according to claim 28, wherein the adjacent chain links are attached to one another by means of at least one fracture element; and wherein, following assembly of the at least one tube with one or more additional tubes, attachment by the at least one fracture element is released by at least one of fracturing, applying fatigue cycles to or melting the at least one fracture element.
